SEQUENCE LISTING (13 sequences)

SEQ ID NO: 1 - protein, 151 aa, Agrostis stolonifera
MSIVRWSNAFDPFSLDLWAEPFDAFRSILPAASGNHDTAAFVNARMDWKETPEAHVFKADLPGVKKEEVKVEVEDGNVLVVSGERSREKEDKNDKWHRVEHSSGKFVRRFRLPENAKVEEVKAGLENGVLTVTVPKAEVKKPEAKAIEISG

SEQ ID NO: 2 - DNA, 456 bp, Agrostis stolonifera
atgtcgatcgtgaggtggagcaacgcgttcgaccccttctccctcgacctctgggcagagcccttcgacgccttccgctccatcctccctgcggcctccggcaaccacgacaccgcagccttcgtgaatgctcgcatggactggaaggagacgccggaagcgcacgtcttcaaagcagacctgcccggcgtgaagaaggaggaggtcaaggtggaggtcgaggatggcaacgtgctcgtcgtcagcggagagcgcagcagggagaaggaggacaagaatgacaagtggcaccgcgtggaacacagctccggcaagtttgtcaggcgcttcaggctgccggagaacgccaaggtggaggaggtgaaggccgggctggagaacggcgtgctcaccgtcaccgtgcccaaggctgaggtcaagaagcccgaggcgaaggccatcgagatctcaggctaa

SEQ ID NO: 3 - protein, 159 aa, Daucus carota
MSIIPSFFGSRRSNVLNPFSLDIWDPFQDYPLITSSGTSSEFGKETAAFANTHIDWKETPQAHVFKADLPGLKKEEVKVEVEEGKVLQISGERNKEKEEKNNKWHRVEFSSGKFLRRFRLPENANVDEVKAGMENGVLTVTVPKVEMKKPEVKSIHISG

SEQ ID NO: 4 - protein, 153 aa, Helianthus annuus
MSIIPSFFTSKRSNIFDPFSLDTWDPFQGIISTEPARETAAIVNARIDWKETPEAHVLKADLPGMKKEEVKVEVEDGRVLQISGERCREQEEKDDTWHRVERSSGKFIRRFRLPENAKMDEVKAMMENGVLTVVVPKEEEEKKPMVKAIDISG

SEQ ID NO: 5 - protein, 157 aa, Arabidopsis thaliana
MSLIPSIFGGRRTNVFDPFSLDVFDPFEGFLTPSGLANAPAMDVAAFTNAKVDWRETPEAHVFKADLPGLRKEEVKVEVEDGNILQISGERSNENEEKNDKWHRVERSSGKFTRRFRLPENAKMEEIKASMENGVLSVTVPKVPEKKPEVKSIDISG

SEQ ID NO: 6 - protein, 162 aa, Tamarix hispida
MSLIPSFFGGLRSNVFDPFSLDVWDPFQGFHFDRSNALSTGVGGDEVSAFVNARMDWKETPGAHIFKADLPGVKKEEVKVEVEDGRVLQITGERSREREEKNDQWHRMERSSGRFMRRFRLPENARTEEVKASMENGVLTVTVPKMEEKKKKPEVKSVAISG

SEQ ID NO: 7 - protein, 143 aa, Medicago sativa
DPFSLDVWDPFKDFPFTNSALSASSFPQENSAFVSTRIDWKETPEAHVFKADLPGLKKEEVKVEIEDDRVLQISGERNVEKEDKNDQWHRVERSSGKFMRRFRLPENAKMDQVKAAMENGVLTVTVPKEEIKKPEVKSIEISS

SEQ ID NO: 8 - protein, 158 aa, Pisum sativum
MSLIPSFFSGRRSNVFDPFSLDVWDPLKDFPFSNSSPSASFPRENPAFVSTRVDWKETPEAHVFKADLPGLKKEEVKVEVEDDRVLQISGERSVEKEDKNDEWHRVERSSGKFLRRFRLPENAKMDKVKASMENGVLTVTVPKEEIKKAEVKSIEISG

SEQ ID NO: 9 - protein, 154 aa, Glycine max
MSLIPSIFGGPRSNVFDPFSLDMWDPFKDFHVPTSSVSAENSAFVNTRVDWKETQEAHVLKADIPGLKKEEVKVQIEDDRVLQISGERNVEKEDKNDTWHRVERSSGKFMRRFRLPENAKVEQVKACMENGVLTVTIPKEEVKKSDVKPIEISG

SEQ ID NO: 10 - protein, 154 aa, Solanum lycopersicum
MSLIPRIFGDRRSSSMFDPFSIDVFDPFRELGFPSTNSGESSAFANTRIDWKETPEPHVFKVDLPGLKKEEVKVEVEEDRVLQISGERNVEKEDKNDKWHRMERSSGKFMRRFRLPENAKMDQVKASMENGVLTVTVPKEEVKKPEVKSIEISG

SEQ ID NO: 11 - protein, 151 aa, Triticum aestivum
MSIVRRSNVFDPFADLWADPFDTFRSIVPAISGGSSETAAFANARVDWKETPEAHVFKVDLPGVKKEEVKVEVEDGNVLVVSGERSREKEDKNDKWHRVERSSGKFVRRFRLPEDAKVEEVKAGLENGVLTVTVPKAEVKKPEVKAIEISG

SEQ ID NO: 12 - protein, 150 aa, Oryza sativa
MSLVRRSNVFDPFSLDLWDPFDSVFRSVVPATSDNDTAAFANARIDWKETPESHVFKADLPGVKKEEVKVEVEEGNVLVISGQRSKEKEDKNDKWHRVERSSGQFMRRFRLPENAKVDQVKAGLENGVLTVTVPKAEVKKPEVKAIEISG

SEQ ID NO: 13 - protein, 152 aa, Zea mays
MSLVRRSNVFDPFSMDLWDPFDTMFRSIVPSAASTNSETAVFASARIDWKETPEAHVFKADLPGVKKEEIKVEVEDGNVLVISGQRSREKEDKDDKWHRVERSSGQFVRRFRLPENAKVDQVKAGLENGVLTVTVPKAEEKKPEVKAIEISG

(19) United States
(12) Patent Application Publication
(10) Pub. No.: US 2015/0337327 A1
(43) Pub. Date: Nov. 26, 2015

(54) Negative Regulator of the Abiotic Stress Response

(71) Applicant: Clemson Univerisity, Clemson, SC (US)

(72) Inventors: Hong Luo, Clemson, SC (US); Xinbo Sun, Clemson, SC (US); Zhigang Li, Clemson, SC (US)

(21) Appl. No.: 14/719,501

(22) Filed: May 22, 2015

Related U.S. Application Data
(60) Provisional application No. 62/002,566, filed on May 23, 2014.

(51) Int. Cl. C12N 15/82; C07K 14/47

(52) U.S. Cl. CPC C12N 15/8271; C07K 14/4702

(57) ABSTRACT

A small heat shock protein, AsHSP17, that has been cloned from creeping bentgrass (Agrostis stolonifera) is described. Transgenic materials incorporating the nucleic acid encoding the small heat shock protein are also described. Also described are methods for modulating a plant's resistance to abiotic stress via manipulation of the expression of the small heat shock protein. For instance, the small heat shock protein can be utilized as a negative regulator of plant response to adverse environmental stresses.

### CROSS REFERENCE TO RELATED APPLICATION

This application claims filing benefit of U.S. Provisional Patent Application Ser. No. 62/002,566, having a filing date of May 23, 2014, which is incorporated herein by reference in its entirety.

### STATEMENT AS TO RIGHTS TO INVENTIONS MADE UNDER FEDERALLY SPONSORED RESEARCH

This invention was made with Government support under competitive grant no. 2010-33522-21656 awarded by the United States Department of Agriculture's National Institute of Food and Agriculture under the Biotechnology Risk Assessment Grant Program and under grant no. CSREES SC-1700450 awarded by the United States Department of Agriculture. The Government has certain rights in the invention.

### SEQUENCE LISTING

The instant application contains a Sequence Listing which has been submitted electronically in ASCII format and is hereby incorporated by reference in its entirety. Said ASCII copy, created on May 19, 2015, is named CXU-817_SL.txt and is 17,481 bytes in size.

### BACKGROUND

Abiotic stresses are environmental stresses that generally restrict growth and/or productivity of plants. Notable abiotic stresses include extremes in temperature, light or other radiation, water availability, and salt levels. A highly conserved set of proteins, the heat shock proteins (HSPs), accumulate when plants are exposed to heat stress. On the basis of their molecular weights, these HSPs have been divided into six classes: HSP100, HSP90, HSP70, HSP60, co-chaperones HSP40 or DNAJ family and small heat shock proteins (sHSPs). sHSPs are the most abundant, ubiquitous and diverse HSP subgroup ranging in size from 12 to 42 kDa. Most sHSPs cannot be detected under normal condition, but are rapidly generated in response to heat stress. It has recently been discovered that accumulation of sHSPs may also be triggered by a variety of other abiotic stresses, though expression cues for different sHSPs can vary. Thus, sHSPs are believed to play an important role in plants' response to abiotic stress.

The sHSP family members have been found in all kingdoms, though plants contain many more members than other organisms. For example, bacteria, archaea and single-celled eukaryotes contain one or two sHSPs while Arabidopsis has 19 sHSPs and rice has 23 sHSPs. Although the sHSP proteins are diverse in both size and sequence, most of them represent some similar characteristic structural feature: a) a highly variable length and sequence N-terminal region, b) a conserved core domain of about 100 amino acids that is homologous to α-crystallin from the vertebrate eye lens (called the α-crystallin domain or small-heat-shock-protein domain) and c) a short C-terminal region. Many studies have demonstrated that most sHSP act as ATP-independent molecular chaperones by bending, unfolding or denaturing proteins and thus protect the cell from damage by preventing irreversible protein aggregation through ATP-dependent systems.

Almost all studies to date show that accumulation of sHSPs has a positive effect on protecting plants against stress. For instance, overexpressing a maize sHSP, ZmHSP16.9, confers tolerance to heat and oxidative stress in transgenic tobacco by increased seed germination rate, root length and oxidant enzyme activities (Sun et al., 2012). Kim et al. (2012) have found that overexpression of OsHSP26 confers enhanced tolerance against oxidative and heat stress in tall fescue (Festuca arudinacea Schreb.). In rice, transgenic plants expressing sHSP17.7 showed heat, drought and UV-B tolerance (Murakami et al., 2004; Sato et al., 2008). In addition, overexpression of a small heat shock protein from Lilium davidii, limhsp16.45, in Arabidopsis enhanced tolerance to abiotic stresses (Mu et al., 2013). Over-accumulation of a chestnut (Castanea sativa) Cl small heat shock protein HSP17.5 enhanced basal thermotolerance in transgenic poplar (Merino et al., 2014). Furthermore, a sHSPs from Rosa chinensis confers resistance to a variety of stresses to E coli, yeast and Arabidopsis thaliana (Jiang et al., 2009). Similarly, overexpression of a heat shock protein (ThHSP18.3) from Tamarix hispida confers stress tolerance to yeast (Gao et al., 2012).

Another study showed that heterologous expression of OsSIZ1, a rice SUMO E3 ligase, enhanced heat tolerance in transgenic creeping bentgrass and also showed that the expression of ApHSP16.5 was significantly more pronounced in the transgenic plants than that in wild type (WT) plants. These results suggest that ApHSP16.5 may play an important role in response to heat stress (Li et al., 2012).

What are needed in the art are additional materials and methods for regulating abiotic stress response in plants.

### SUMMARY

According to one embodiment, disclosed is an isolated nucleic acid molecule comprising a synthetic polynucleotide that encodes a small heat shock protein as set forth in SEQ ID NO: 1 or a polynucleotide that encodes a functional equivalent of SEQ ID NO: 1. For instance, the isolated nucleic acid molecule can include the nucleic acid sequence as set forth in SEQ ID NO: 2 or a polynucleotide that hybridizes to SEQ ID NO: 2. In one embodiment the isolated nucleic acid molecule can include a polynucleotide that is complementary to SEQ ID NO: 2. As utilized herein, the term “synthetic polynucleotide” generally refers to a polynucleotide that differs in some fashion from a naturally occurring polynucleotide, e.g., a cDNA or other variation from a polynucleotide occurring in nature.

Also disclosed is an expression cassette including one of these polynucleotides and a host cell including the expression cassette. Transgenic plants and progeny thereof as well as transgenic seeds including the expression cassette are also disclosed.

Methods for altering the abiotic stress response of a plant are also described. For instance, the methods can include down regulating the expression of the small heat shock protein, AsHSP17 (SEQ ID NO: 1) in a plant, e.g., a monocot, so as to increase the plant's resistance to abiotic stress.

### BRIEF DESCRIPTION OF THE FIGURES

The present disclosure may be better understood with reference to the figures including:

FIG. 1 is a comparison of the amino acid alignment for AsHSP17 (SEQ ID NO: 1) with other plant short heat shock proteins including Daucus carota HSP18.0 (SEQ ID NO: 3; GenBank Accession No. X53852); Helianthus annuus HSP17.6 (SEQ ID NO: 4; GenBank Accession No. X59701); Arabidopsis thaliana HSP17.6 (SEQ ID NO: 5; GenBank Accession No. X16076); Tamarix hispida HSP26 (SEQ ID NO: 6; GenBank Accession No. JX482112); Medicago sativa HSP18.1 (SEQ ID NO: 7; GenBank Accession No. X58710); Pisum sativum HSP18.1 (SEQ ID NO: 8; GenBank Accession No. M33899); Glycine max HSP17.6 (SEQ ID NO: 9; GenBank Accession No.MI1317); Solanum lycopersicum HSP17.8 (SEQ ID NO: 10; GenBank Accession No. X56138); Agrostis stolonifera HSP17 (SEQ ID NO: 1); Triticum aestivum HSP16.9A (SEQ ID NO: 11; GenBank Accession No. X13431); Oryza sativa HSP16.9A (SEQ ID NO: 12; GenBank Accession No. X60820); Zea mays HSP16.9 (SEQ ID NO: 13; GenBank Accession No. 13 NM_{—}001157311)

FIG. 2 presents the phylogenetic relationship of AsHSP17 (SEQ ID NO: 1) with different classes of sHSP from other plant species.

FIG. 3 illustrates localization of AsHSP17 protein within the protoplast by GFP assay. WT, Wild type; 35S-GFP, control with empty vector; AsHSP17-GFP, AsHSP17-GFP fusion. Bars=10 μm.

FIG. 4A presents the semi-quantitative RT-PCR analysis of the expression levels of AsHSP17 under heat, salt, drought and ABA treatment.

FIG. 4B presents the real-time quantitative RT-PCR analysis of expression of AsHSP17 under heat treatment.

FIG. 4C presents the real-time quantitative RT-PCR analysis of expression of AsHSP17 under salt treatment.

FIG. 4D presents the real-time quantitative RT-PCR analysis of expression of AsHSP17 under drought treatment.

FIG. 4E presents the real-time quantitative RT-PCR analysis of expression of AsHSP17 under ABA treatment.

FIG. 5A is a schematic diagram of the AsHSP17 chimeric gene expression construct, p35S: AsHSP17/p35S: bar. The AsHSP17 gene and a herbicide resistance gene, bar, are under control of the CaMV 35S promoter separately. RB, right border; LB, left border.

FIG. 5B presents the PCR analysis of AsHSP17 and bar gene in wild type (WT) and TG plants to detect transgene insertion in Arabidopsis thaliana genome.

FIG. 5C presents the expression analysis of AsHSP17 (SEQ ID NO: 2) in wild type (WT) and transgenic (TG) plants transcription.

FIG. 5D presents Real-time RT-PCR analysis of the expression of the AsHSP17 in WT and TG plants. The AACt method was used for real time PCR analysis. Creeping bentgrass ubiquitin gene AsUBQ was used as the endogenous control. Error bars indicate SE (n=3).

FIG. 6A illustrate two week old Arabidopsis thaliana WT and four TG lines grown under normal condition in growth chamber.

FIG. 6B illustrates the plants following the application of heat stress by heating the plants to 40° C. for 2 days. The plants were then moved back to normal condition. Photographs were taken at 2 days (FIG. 6C) and 4 days (FIG. 6D) after being moved back to normal conditions. Leaf sample collected after 2 days of heat treatment were used for measuring electrolyte leakage (FIG. 6E), relative water content (FIG. 6F) (n=3).

FIG. 6G presents the chlorophyll a content from the leaf samples after 2 days of heat treatment (n=3).

FIG. 6H presents the chlorophyll b content from the leaf samples after 2 days of heat treatment (n=3).

FIG. 6I presents the total chlorophyll content from the leaf samples after 2 days of heat treatment (n=3).

FIG. 6J presents the chlorophyll a content for the leaf samples under normal conditions and 3 days after salt (175 mM NaCl) treatment (n=3).

FIG. 6K presents the chlorophyll b content for the leaf samples under normal conditions and 3 days after salt (175 mM NaCl) treatment (n=3).

FIG. 6L presents the total chlorophyll content for the leaf samples under normal conditions and 3 days after salt (175 mM NaCl) treatment (n=3).

FIG. 6M presents the photosynthesis rate.

FIG. 6N presents the transpiration rate

FIG. 6O presents the Stomatal conductance of wild type (WT) and transgenic (TG) plants under normal growth conditions (n=9).

FIG. 6P presents the expression of five photosynthesis-related genes in wild type (WT) and transgenic (TG) plants under normal growth conditions.

FIG. 7A illustrates the germination of wild-type (WT) and AsHSP17 transgenic plant seeds in 0 mM, 125 mM, 150 mM and 175 mM of NaCl after 6d.

FIG. 7B presents the termination percentage of wild-type (WT) and transgenic (TG) seeds on 0, 125 mM, 150 mM and 175 mM NaCl 4 d after treatment (n=3).

FIG. 7C presents the greening percentage of wild-type (WT) and transgenic (TG) seeds on 0, 125 mM, 150 mM and 175 mM NaCl 4 d after treatment (n=3).

FIG. 7D presents the germination percentage of wild-type (WT) and transgenic (TG) seeds on 0, 125 mM, 150 mM and 175 mM NaCl 6 d after treatment (n=3).

FIG. 7E presents the greening percentage of wild-type (WT) and transgenic (TG) seeds on 0, 125 mM, 150 mM and 175 mM NaCl 6 d after treatment (n=3).

FIG. 8A illustrates seed germination of wild-type (WT) and AsHSP17 transgenic plants subjected to 0, 0.75 and 1 μM of ABA 4, 6, 8 and 10 d after treatment.

FIG. 8B presents germination percentages of wild type (WT) and transgenic (TG) seeds subjected to 0, 0.75 1 μM of ABA 6 d after treatment (n=3).

FIG. 8C presents greening percentages of wild type (WT) and transgenic (TG) seeds subjected to 0, 0.75 and 1 μM of ABA 6 d after treatment (n=3).

FIG. 9A presents the relative expression profile of heat stress-responsive gene HSFA1b in 3 weeks old WT and AsHSP17 TG plants subjected to heat stress at 40° C. for 0 or 4 h.

FIG. 9B presents the relative expression profile of heat stress-responsive gene HSFA1d in 3 weeks old WT and AsHSP17 TG plants subjected to heat stress at 40° C. for 0 or 4 h.

FIG. 9C presents the relative expression profile of heat stress-responsive gene HSFA2 in 3 weeks old WT and AsHSP17 TG plants subjected to heat stress at 40° C. for 0 or 4 h.

FIG. 9D presents the relative expression profile of heat stress-responsive gene HSFA3 in 3 weeks old WT and AsHSP17 TG plants subjected to heat stress at 40° C. for 0 or 4 h.

FIG. 9E presents the relative expression profile of heat stress-responsive gene HSFB1 in 3 weeks old WT and AsHSP17 TG plants subjected to heat stress at 40° C. for 0 or 4 h.

FIG. 9F presents the relative expression profile of heat stress-responsive gene HSP17.6A in 3 weeks old WT and AsHSP17 TG plants subjected to heat stress at 40° C. for 0 or 4 h.

FIG. 9G presents the relative expression profile of heat stress-responsive gene HSP90-1 in 3 weeks old WT and AsHSP17 TG plants subjected to heat stress at 40° C. for 0 or 4 h.

FIG. 9H presents the relative expression profile of heat stress-responsive gene HSP101 in 3 weeks old WT and AsHSP17 TG plants subjected to heat stress at 40° C. for 0 or 4 h.

FIG. 10A illustrates two weeks old Arabidopsis thaliana WT and three TG lines were grown under normal conditions in growth chamber and subjected to 175 mM NaCl treatment, and photographed 0 and 10 d after treatment and 4 d after recovery.

FIG. 10B presents the electrolyte leakage of leaf samples collected 3 d after salt treatment (n=3).

FIG. 10C presents the relative water content of leaf samples collected 3 d after salt treatment (n=3).

FIG. 10D presents the expression profile of salt stress-responsive gene ADC1 in 3 weeks old wild type (WT) and AsHSP17 transgenic (TG) plants subjected to salt stress (200 mM NaCl) for 0 or 4 h.

FIG. 10E presents the expression profile of salt stress-responsive gene SAMDC in 3 weeks old wild type (WT) and AsHSP17 transgenic (TG) plants subjected to salt stress (200 mM NaCl) for 0 or 4 h.

FIG. 10F presents the expression profile of salt stress-responsive gene SPDS1 in 3 weeks old wild type (WT) and AsHSP17 transgenic (TG) plants subjected to salt stress (200 mM NaCl) for 0 or 4 h.

Expression profiles of ABA stress-responsive genes including NCED3 (FIG. 11A), CYP707A3 (FIG. 11B), Rab18 (FIG. 11C), ABF2 (FIG. 11D), COR47 (FIG. 11E), RD29A (FIG. 11F), AB13 (FIG. 11G), AB14 (FIG. 11H), and AB15 (FIG. 11I) in 3 weeks old wild type (WT) and AsHSP17 transgenic (TG) plants subjected to 50 μM ABA for 0 or 4 h. The ΔΔCt method was used for real time PCR analysis. Actin gene AtActin1 was used as the endogenous control. Error bars represent SE.

Expression profiles of stress-related genes including DREBIA (FIG. 12A), DREB1B (FIG. 12B), DREB2A (FIG. 12C), DREB2B (FIG. 12D), NAC019 (FIG. 12E), NAC072 (FIG. 12F), ERF53 (FIG. 12G), miR396a (FIG. 12H), miR156c (FIG. 12I), miR156e (FIG. 12J) in 3 weeks old wild type (WT) and AsHSP17 transgenic (TG) plants subjected to heat (40° C.), salt (200 mM NaCl) and 50 μM ABA for 0 or 4 h. The ΔΔCt method was used for real time PCR analysis. Actin gene AtActin1 was used as the endogenous control. Error bars represent SE.

Repeat use of reference characters in the present specification and drawings is intended to represent the same or analogous features or elements of the present disclosure.

### DETAILED DESCRIPTION

Reference will now be made in detail to various embodiments of the disclosure, one or more examples of which are illustrated in the accompanying drawings. Each example is provided by way of explanation of the subject matter, not limitation thereof. In fact, it will be apparent to those skilled in the art that various modifications and variations can be made in the present disclosure without departing from the scope or spirit of the subject matter. For instance, features illustrated or described as part of one embodiment, can be used on another embodiment to yield a still further embodiment.

The present disclosure generally relates to a small heat shock protein, AsHSP17 (SEQ ID NO: 1) that has been cloned from creeping bentgrass (Agrostis stolonifera) and methods for affecting a plant's resistance to abiotic stress via manipulation of the expression of the small heat shock protein. The cDNA AsHSP17 sequence is provided in SEQ ID NO: 2, which encodes the protein (SEQ ID NO: 1) of 17 kDa and an isoelectric oint of approximately 5.87.

The expression of AsHSP17 can be strongly induced in both leaf and root tissues by heat treatment. AsHSP17 expression can also be induced somewhat in root by salt and abscisic acid (ABA) treatment, but not by water withholding. AsHSP17 expression is slight or non-existent in leaf tissue under salt, drought and ABA treatment. These results indicate that AsHSP17 accumulates in response to heat, salt and ABA.

In contrast to the function of most known sHSPs, overexpression of AsHSP17 under abiotic stress conditions can lead to higher sensitivity to stress in the plant. Overexpression of AsHSP17 can also lead to decreased seed germination under salt and ABA treatment. Thus, AsHSP17 can be utilized as a negative regulator of plant response to adverse environmental stresses.

Accordingly, in one aspect, the present disclosure is directed to modulation of the expression of AsHSP17 in plants. The term “plant” means any plant and thus can include, without limitation, angiosperms, gymnosperms, bryophytes, ferns and/or fem allies.

According to one embodiment, AsHSP17 expression can be down regulated, which can provide improved plant resistance to various abiotic stresses (e.g., temperature and/or salination) in desirable species. Desirable species can include, for example turf grasses, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, chinese cabbage, bolt choy), cardoni, carrots, napa, okra, onions, celery, parsley, chick peas, parsnips, chicory, peppers, potatoes, cucurbits (e.g., marrow, cucumber, zucchini, squash, pumpkin), radishes, dry bulb onions, rutabaga, eggplant, salsify, escarole, shallots, endive, garlic, spinach, green onions, squash, greens, beet (sugar beet and fodder beet), sweet potatoes, swiss chard, horseradish, tomatoes, turnips, and spices; a fruit and/or vine crop such as apples, apricots, cherries, nectarines, peaches, pears, plums, prunes, cherry, quince, almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pomes, melon, mango, papaya, and lychee, a field crop plant such as clover, alfalfa, evening primrose, meadow foam, corn/maize (field, sweet, popcomrn), hops, jojoba, peanuts, rice, safflower, small grains (barley, oats, rye, wheat, etc.), sorghum, tobacco, kapok, a leguminous plant (beans, lentils, peas, soybeans), an oil plant (rape, mustard, poppy, olive, sunflower, coconut, castor oil plant, cocoa bean, groundnut), Arabidopsis, a fibre plant (cotton, flax, hemp, jute), lauraceae (cinnamon, camphor), or a plant such as coffee, sugar cane, tea, and natural rubber plants; and/or a bedding plant such as a flowering plant, a cactus, a succulent and/or an omamental plant, as well as trees such as forest (broad-leaved trees and evergreens, such as conifers), fruit, omamental, and nut-bearing trees, as well as shrubs and other nursery stock.

In particular embodiments, a plant cell and/or plant is a turfgrass. Turfgrass can include, but is not limited to, Sporobolus airiodes, Puccinellia distans, Paspalum notatum, Cynodon dactylon, Buchloe dactyloides, Cenchrus ciliaris, Hordeum califormicum, Hordeum vulgare, Hordeum brachyantherum, Agrostis capillaries, Agrostis stolinifera, Agrostis exerata, Briza maxima, Poa annua, Poe ample, Poe canbyi, Poe compressa, Poa pratensis, Poa scabrella, Poe trivialis, Poe secunda, Andropogon gerardii, Schizachyruim scoparium, Andropogon hallii, Bromus arizonicus, Bromus carinatus, Bromus biebersteinii, Bromus marginatus, Bromus rubens, Bromus inermis, Buchloe dactyloides, Axonopus fussifolius, Eremochloa ophiuroides, Muhlenbergia rigens, Sporobolus cryptandrus, Sporobolus heterolepis, Tripsacum dactyloides, Festuce arizonica, Festuca rubra var. commutate, Festuca rubra var. rubra, Festuca megalura, Festuca longifolia, Festuca idahoensis, Festuca elation, Fescue rubra, Fescue ovina var. ovina, Festuca arundinacea, Alopecurus arundinaceaus, Alopecurus pratensis, Hilaria jamesii, Bouteloua eriopoda, Bouteloua gracilis, Bouteloua curtipendula, Deschampsia caespitosa, Oryzopsis hymenoides, Sorghastrum nutans, Eragrostis trichodes, Eragrostis curvula, Melice californica, Stipa comate, Stipa lepida, Stipa viridulea, Stipa cemua, Stipa pulchra, Dactylis glomerata, Koeleria pyramidata, Calamovilfa longifolia, Agrostis alba, Phalaris arundinacea, Stenotaphrum secundatum, Spartina pectinata, Lolium multiflorum, Lolium perenne, Leptochloa dubia, Sitanion hystrix, Panicum virgatum, Aristida purpurea, Phleum pretense, Agropyron spicatum, Agropyron cristatum, Agropyron desertorum, Agropyron intermedium, Agropyron trichophorum, Agropyron trachycaulum, Agropyron riparium, Agropyron elongatum, Agropyron smithii, Elymus glaucus, Elymus Canadensis, Elymus triticoides, Elymus junceus, Zoysia japonica, Zoysia matrella, and Zoysia tenuifolia. In some embodiments, a plant of the present invention is creeping bent grass, Agrostis palustris.

In another embodiment, AsHSP17 expression can be up regulated or induced, which can provide decreased plant resistant to various abiotic stresses in non-desirable species, for instance weeds and invasive species. Non-desirous species (e.g., weeds) that can be transformed to upregulate or induce expression of asHSP17 can include, without limitation, crabgrass (Digitaria sp.), dandelion (Taraxacum officinale W), smartweed (Polygonum sp.), redroot pigweed (Amaranthus retroflexus L), purslane (Portulaca oleracea L), lambsquarters (Chenopodium album L), foxtail (Setaria sp.), bamyardgrass (Echinochloa crus-galli (L.) Beauv), spiny amaranth (A. spinosus), Palmer amaranth (A. palmen), smooth pigweed (A. hybridus), tall waterhemp (A. tuberculatus), tumble pigweed (A. albus), livid amaranth (A. lividus), slender amaranth (A. viridus), powell amaranth (A. powellii), pitted morning glory (lpomoea lacunosa), smallflower morning glory (Jacquemontia tamnifolia), entire leaf morning glory (Ipomoea hederacea), ivyleaf morning glory (Ipomoea hederacea), tall morning glory (Ipomoea purpurea), palmleaf morning glory (Ipomoea wrightii), prickly sida (Sida spinosa), velvetleaf (Abutilon theophrasti), spurred anoda (Anoda cristata), sicklepod (Cassia obtusifolia), hemp sesbania (Sesbania exaltata), common purslane (Portulaca oleracea), carpetweed (Mollugo verticillata), Florida purslant (Richardia scabra), annual smartweeds (Polygonum spp.), Florida beggarweed (Desmodium tortuosum), common ragweed (Ambrosia artemisifolia), coffee senna (Cassia occidentalis), redweed (Melochia corchorifolia), black night shade (Solanum nigrum), jimsonweed (Datura stramonium), kochia (Kochia scoparia), wild radish (Raphanus raphanistrum), giant foxtail (Setaria faberi), green foxtail (Seteria viridis), yellow foxtail (Setaria glauca), barnyardgrass (Echinochloa crus-galli), jungle rice (Echinochloa colonum), large crabgrass (Digitaria sanguinalis), smooth crabgrass (Digitaria ischaemum), fall panicum (Panicum dichotomiflorum), annual sedges (Cyperus spp.), spotted spurge (Euphorbia maculata), common cocklebur (Xanthium strumarium); wild mustard, broadleaf signalgrass (Brachiaria platyphylla), goosegrass (Eleusine indica), yellow nutsedge (Cyperus esculentus), purple nutsedge (Cyperus rotundus), sprangletops (Leptochloa spp.), crowfootgrass (Dactyloctenium aegyptium), Texas panicum (Panicum texanum), large flowered evening primrose (Oenothera erythrosepala), common evening primrose (Oenothera biennis), cutleaf evening primrose (Oenothera laciniata), and so forth.

According to one embodiment, disclosed is an isolated polynucleotide comprising a coding region that encodes AsHSP17 or a functional equivalent thereof. For example, the coding region can include the cDNA sequence of SEQ ID NO: 2.

The present disclosure also relates to a recombinant polynucleotide that includes an AsHSP17 nucleotide sequence as disclosed herein or functional portion thereof operatively linked to a heterologous nucleotide sequence. For instance, the heterologous nucleotide sequence can be one that is not present in conjunction with the AsHSP17 nucleotide sequence in a naturally occurring plant. In another embodiment, the recombinant polynucleotide can include a coding region disclosed herein, or a functional portion thereof, of AsHSP17 coding sequence operatively linked to a heterologous promoter. The heterologous promoter can provide a means to express AsHSP17 constitutively, inducibly, or in a tissue-specific or phase-specific manner.

One aspect of the present disclosure provides a method for determining whether a test plant, for example a monocot, has been exposed to at least one stress condition, for example an abiotic stress, and more particularly heat. For example, the method can include determining a polynucleotide expression in the test plant and comparing the polynucleotide expression of the test plant to the expression of at least one reference plant that has been exposed to at least one abiotic stress, for example, heat. In one embodiment the expressed polynucleotide can be SEQ ID NO: 2.

Another aspect provides an isolated nucleic acid sequence comprising a plant nucleotide sequence of at least 10 nucleotides long that hybridizes to the complement of SEQ ID NO: 2, or a functional portion thereof, which is operably linked to a regulatory element or functional portion thereof. In one embodiment, the regulatory element or functional portion thereof alters transcription of an operatively linked nucleic acid sequence in response to an abiotic stress.

Hybridization conditions can be, for example:

(i) 7% sodium dodecyl sulfate (SDS), 0.5 M NaPO₄, 1 mM ethylenediamine tetraacetic acid (EDTA) at 50° C. with a final wash in 2× standard saline citrate (SSC), 0.1% SDS at 50° C.;

(ii) 7% SDS, 0.5 M NaPO₄, 1 mM EDTA at 50° C. with a final wash in 1×SSC, 0.1% SDS at 50° C.;

(iii) 7% SDS, 0.5 M NaPO₄, 1 mM EDTA at 50° C. with a final wash in 0.5×SSC, 0.1% SDS at 50° C.;

(iv) 7% SDS, 0.5 M NaPO₄, 1 mM EDTA at 50° C. with a final wash in 0.1×SSC, 0.1% SDS at 50° C.; and

(v) 7% SDS, 0.5 M NaPO₄, 1 mM EDTA at 50° C. with a final wash in 0.1×SSC, 0.1% SDS at 65° C.

When hybridization is performed under stringent conditions, either the test or nucleic acid molecule of presently disclosed subject matter can be present on a support: e.g., on a membrane or on a DNA chip. Thus, either a denatured test or nucleic acid molecule of the presently disclosed subject matter is first bound to a support and hybridization is effected for a specified period of time under conditions as described above.

One specific embodiment is directed to an isolated nucleic acid molecule comprising a polynucleotide selected from the group consisting of: a) SEQ ID NO: 2; b) a functional portion of any of the SEQ ID NO: 2; c) a polynucleotide that is substantially similar to a sequence SEQ ID NO: 2 or a functional portion of SEQ ID NO: 2; d) a sequence of at least 15 nucleotides that hybridizes under any of the hybridization conditions (i), (ii), (iii), (iv) or (v) to a polynucleotide of a), b) or c); e) the complement of any sequence of a), b), c) or d); f) the reverse complement of any sequence of a), b), c) or d); and g) an allelic variant of any of the above.

Also provided are expression cassettes, plants and seeds comprising any of the disclosed isolated sequences.

According to another embodiment, disclosed is a method of producing a transgenic plant that includes at least one plant cell that exhibits altered responsiveness to a stress condition, particularly an abiotic stress, and more particularly heat and/or osmotic stress. In one embodiment, the method can be performed by introducing a nucleotide sequence comprising SEQ ID NO: 2 or a functional portion of SEQ ID NO: 2 into a plant cell genome, whereby the nucleotide sequence modulates a response of the plant cell to a stress condition. The functional portion of SEQ ID NO: 2 can encode SEQ ID NO: 1 or functional peptide portion thereof, wherein expression of the SEQ ID NO: 1 or functional peptide portion thereof decreases the stress tolerance of the transgenic plant. The nucleotide sequence can also include SEQ ID NO: 2 or a functional portion thereof operatively linked to a heterologous promoter. The nucleotide sequence can integrate into the plant cell genome in a site-specific manner, whereupon it can be operatively linked to a heterologous nucleotide sequence, which can be expressed in response to a stress condition specific for the regulatory element; or can be a mutant regulatory element, which is not responsive to the stress condition, whereby upon integrating into the plant cell genome, the mutant regulatory element disrupts an endogenous stress-regulated regulatory element of a plant stress-regulated nucleotide sequence, thereby altering the responsiveness of the plant stress-regulated nucleotide sequence to the stress condition.

According to another embodiment, disclosed are methods for down regulating expression of SEQ ID NO: 1 or a functional equivalent thereof. In one embodiment, the method can be performed by introducing a coding sequence into a plant genome, for instance via an expression cassette. The coding region of the expression cassette can include sequences encoding sequences involved in gene silencing such as antisense sequences, double stranded RNAi sequences, a triplexing agent, and sequences comprising dominant negative mutations. In additional related aspects, the coding regions comprise sequences encoding polypeptides that alter the response of a plant to an abiotic stress.

Further aspects include plants and uniform populations of plants made by the above methods as well as seeds and progeny from such plants and cDNA or genomic DNA libraries prepared from the transgenic plant, or from a plant cell from said transgenic plant, wherein said plant cell exhibits altered responsiveness to the stress condition.

One aspect of the presently disclosed subject matter provides compositions and methods for modulating (i.e. increasing or decreasing) the level of nucleic acid molecules and/or polypeptides of the presently disclosed subject matter in plants. In particular, the nucleic acid molecules and polypeptides of the presently disclosed subject matter are expressed constitutively, temporally, or spatially (e.g., at developmental stages), in certain tissues, and/or quantities, which are uncharacteristic of non-recombinantly engineered plants. Therefore, the presently disclosed subject matter provides utility in such exemplary applications as altering the specified characteristics identified above.

Modification (i.e. increasing or decreasing) the concentration or composition of AsHSP17 can be effected by increasing or decreasing the concentration and/or the composition (i.e. the ration of the polypeptides of the presently disclosed subject matter) in a plant. The method comprises introducing into a plant cell an expression cassette comprising a nucleic acid molecule of the presently disclosed subject matter as disclosed above to obtain a transformed plant cell or tissue, and culturing the transformed plant cell or tissue. The nucleic acid molecule can be under the regulation of a constitutive or inducible promoter. The method can further comprise inducing or repressing expression of a nucleic acid molecule of a sequence in the plant for a time sufficient to modify the concentration and/or composition in the plant or plant part.

A plant or plant part having modified expression of a nucleic acid molecule of the presently disclosed subject matter can be analyzed and selected using methods known to those skilled in the art including, but not limited to, Southern blotting, DNA sequencing, or PCR analysis using primers specific to the nucleic acid molecule and detecting amplicons produced therefrom.

In general, a concentration or composition is increased or decreased by at least in one embodiment 5%, in another embodiment 10%, in another embodiment 20%, in another embodiment 30%, in another embodiment 40%, in another embodiment 50%, in another embodiment 60%, in another embodiment 70%, in another embodiment 80%, and in still another embodiment 90% relative to a native control plant, plant part, or cell lacking the expression cassette.

Increasing the level of expression of AsHSP17 in a cell can be accomplished by transforming the cell with a nucleic acid molecule encoding the protein according to standard methods such as those described herein.

Reducing the level of expression of AsHSP17 in a cell can likewise be accomplished using standard methods. For example, an antisense RNA or DNA oligonucleotide that is complementary to the sense strand (i.e., the mRNA strand or cDNA strand) of a nucleic acid molecule encoding the protein can be administered to the cell to reduce expression of that protein in that cell (see e.g., Agrawal, 1993; U.S. Pat. No. 5,929,226, which is incorporated herein by reference).

The modulation in expression of AsHSP17 can be achieved, for example, in one of the following ways:

### 1. “Sense” Suppression

Alteration of the expression of a nucleotide sequence of AsHSP17, in one embodiment reduction of its expression, can be obtained by “sense” suppression (referenced in e.g., Jorgensen et al., 1996). In this case, the entirety or a portion of a nucleotide sequence of the presently disclosed subject matter is comprised in a DNA molecule. The DNA molecule can be operatively linked to a promoter functional in a cell comprising the target gene, in one embodiment a plant cell, and introduced into the cell, in which the nucleotide sequence is expressible. The nucleotide sequence is inserted in the DNA molecule in the “sense orientation”, meaning that the coding strand of the nucleotide sequence can be transcribed. In one embodiment, the nucleotide sequence is fully translatable and all the genetic information comprised in the nucleotide sequence, or portion thereof, is translated into a polypeptide. In another embodiment, the nucleotide sequence is partially translatable and a short peptide is translated. In one embodiment, this is achieved by inserting at least one premature stop codon in the nucleotide sequence, which brings translation to a halt. In another embodiment, the nucleotide sequence is transcribed but no translation product is made. This is usually achieved by removing the start codon, i.e. the “ATG”, of the polypeptide encoded by the nucleotide sequence. In a further embodiment, the DNA molecule comprising the nucleotide sequence, or a portion thereof, is stably integrated in the genome of the plant cell. In another embodiment, the DNA molecule comprising the nucleotide sequence, or a portion thereof, is comprised in an extrachromosomally replicating molecule.

In transgenic plants containing one of the DNA molecules disclosed herein, the expression of the nucleotide sequence corresponding to the nucleotide sequence comprised in the DNA molecule can be reduced. The nucleotide sequence in the DNA molecule in one embodiment is at least 70% identical to the nucleotide sequence the expression of which is reduced, in another embodiment is at least 80% identical, in another embodiment is at least 90% identical, in another embodiment is at least 95% identical, and in still another embodiment is at least 99% identical.

### 2. “Antisense” Suppression

In another embodiment, the alteration of the expression of AsHSP17, for example the reduction of its expression, is obtained by “antisense” suppression. The entirety or a portion of a nucleotide sequence is comprised in a polynucleotide molecule. The molecule can be operatively linked to a promoter functional in a plant cell, and introduced in a plant cell, in which the nucleotide sequence is expressible. The nucleotide sequence is inserted in the DNA molecule in the “antisense orientation”, meaning that the reverse complement (also called sometimes non-coding strand) of the nucleotide sequence can be transcribed. In one embodiment, the DNA molecule comprising the nucleotide sequence, or a portion thereof, is stably integrated in the genome of the plant cell. In another embodiment the DNA molecule comprising the nucleotide sequence, or a portion thereof, is comprised in an extrachromosomally replicating molecule. Several publications describing this approach are cited for further illustration (Green et al., 1986; van der Krol et al., 1991; Powell et al., 1989; Ecker & Davis, 1986).

### 3. Homologous Recombination

In another embodiment, at least one genomic copy corresponding to AsHSP17 is modified in the genome of the plant by homologous recombination as further illustrated in Paszkowski et al., 1988. This technique uses the ability of homologous sequences to recognize each other and to exchange nucleotide sequences between respective nucleic acid molecules by a process known in the art as homologous recombination. Homologous recombination can occur between the chromosomal copy of a nucleotide sequence in a cell and an incoming copy of the nucleotide sequence introduced in the cell by transformation. Specific modifications are thus accurately introduced in the chromosomal copy of the nucleotide sequence. In one embodiment, the regulatory elements of the nucleotide sequence are modified. Such regulatory elements are easily obtainable by screening a genomic library using the nucleotide sequence of the presently disclosed subject matter, or a portion thereof, as a probe. The existing regulatory elements are replaced by different regulatory elements, thus altering expression of the nucleotide sequence, or they are mutated or deleted, thus abolishing the expression of the nucleotide sequence. In another embodiment, the nucleotide sequence is modified by deletion of a part of the nucleotide sequence or the entire nucleotide sequence, or by mutation. Expression of a mutated polypeptide in a plant cell is also provided in the presently disclosed subject matter. Recent refinements of this technique to disrupt endogenous plant genes have been disclosed (Kempin et al., 1997 and Miao & Lam, 1995).

In one embodiment, a mutation in the chromosomal copy of a nucleotide sequence is introduced by transforming a cell with a chimeric oligonucleotide composed of a contiguous stretch of RNA and DNA residues in a duplex conformation with double hairpin caps on the ends. An additional feature of the oligonucleotide is for example the presence of 2′-O-methylation at the RNA residues. The RNA/DNA sequence is designed to align with the sequence of a chromosomal copy of a nucleotide sequence of the presently disclosed subject matter and to contain the desired nucleotide change. For example, this technique is further illustrated in U.S. Pat. No. 5,501,967 and Zhu et al., 1999, incorporated herein by reference.

### 4. Ribozymes

In a further embodiment, an RNA coding for a polypeptide of AsHSP17 can be cleaved by a catalytic RNA, or ribozyme, specific for such RNA. The ribozyme is expressed in transgenic plants and results in reduced amounts of RNA coding for the polypeptide in plant cells, thus leading to reduced amounts of polypeptide accumulated in the cells. This method is further illustrated in U.S. Pat. No. 4,987,071, incorporated herein by reference.

### 5. Dominant-Negative Mutants

In another embodiment, the activity of an AsHSPI7 peptide is changed. This is achieved by expression of dominant negative mutants of the polypeptides in transgenic plants, leading to the loss of activity of the endogenous polypeptide.

### 6. Aptamers

In a further embodiment, the activity of AsHSP17 can be inhibited by expressing in transgenic plants nucleic acid ligands, so-called aptamers, which specifically bind to the polypeptide. Aptamers can be obtained by the SELEX (Systematic Evolution of Ligands by Exponential Enrichment) method. In the SELEX method, a candidate mixture of single stranded nucleic acids having regions of randomized sequence is contacted with the polypeptide and those nucleic acids having an increased affinity to the target are partitioned from the remainder of the candidate mixture. The partitioned nucleic acids are amplified to yield a ligand-enriched mixture. After several iterations a nucleic acid with optimal affinity to the polypeptide is obtained and is used for expression in transgenic plants. This method is further illustrated in U.S. Pat. No. 5,270,163, incorporated herein by reference.

### 7. Engineered Nucleases

A nuclease that binds a nucleotide sequence of AsHSP17 or to its regulatory region can also be used to alter expression of the nucleotide sequence. In altemative embodiments, transcription of the nucleotide sequence is reduced or increased. Engineered nucleases as may be utilized can include, without limitation, zinc finger nucleases (ZFNs), Transcription Activator-Like Effector Nucleases (TALENs), the CRISPR/Cas system, and engineered meganuclease re-engineered homing endonucleases. Zinc finger polypeptides are disclosed in, for example, Beerli et al., 1998, or in WO 95/19431, WO 98/54311, or WO 96/06166, all incorporated herein by reference in their entirety. TALENs have been described by Singh, et al. (Joumal of Biological Sciences, 13)3): 91-94, 2013) and are disclosed in Voytas, et al. (US Patent Application Publication No. 2011/0145940), incorporated herein by reference. CRISPR/Cas systems are described in, for example, Horvath, et al.

(Science 327 (5962): 167-170) and Marraffini, et al. (Nature Reviews Geneticsl 11 (3): 181-190) and by Doudna, et al. (U.S. Patent Application Publication No. 2014/0068797), incorporated herein by reference.
8. dsRNA

Alteration of the expression of AsHSP17 can also be obtained by double stranded RNA (dsRNA) interference (RNAi) as disclosed, for example, in WO 99/32619, WO 99/53050, or WO 99/61631, all incorporated herein by reference in their entireties. In one embodiment, the alteration of the expression of AsHSP17, in one embodiment the reduction of its expression, is obtained by dsRNA interference. The entirety, or in one embodiment a portion, of a nucleotide sequence of AsHSP17 can be comprised in a DNA molecule. The size of the DNA molecule is in one embodiment from 100 to 1000 nucleotides or more; the optimal size to be determined empirically. Two copies of the identical DNA molecule are linked, separated by a spacer DNA molecule, such that the first and second copies are in opposite orientations. In one embodiment, the first copy of the DNA molecule is the reverse complement (also known as the non-coding strand) and the second copy is the coding strand; in another embodiment, the first copy is the coding strand, and the second copy is the reverse complement. The size of the spacer DNA molecule is in one embodiment 200 to 10,000 nucleotides, in another embodiment 400 to 5000 nucleotides, and in yet another embodiment 600 to 1500 nucleotides in length. The spacer is in one embodiment a random piece of DNA, in another embodiment a random piece of DNA without homology to the target organism for dsRNA interference, and in still another embodiment a functional intron that is effectively spliced by the target organism. The two copies of the DNA molecule separated by the spacer are operatively linked to a promoter functional in a plant cell, and introduced in a plant cell in which the nucleotide sequence is expressible. In one embodiment, the DNA molecule comprising the nucleotide sequence, or a portion thereof, is stably integrated in the genome of the plant cell. In another embodiment, the DNA molecule comprising the nucleotide sequence, or a portion thereof, is comprised in an extrachromosomally replicating molecule. Several publications describing this approach are cited for further illustration (Waterhouse et al., 1998; Chuang & Meyerowitz, 2000; Smith et al., 2000).

In another non-limiting example, RNA interference (RNAi) or post-transcriptional gene silencing (PTGS) can be employed to reduce the level of expression of AsHSP17 in a cell. As used herein, the terms “RNA interference” and “post-transcriptional gene silencing” are used interchangeably and refer to a process of sequence-specific modulation of gene expression mediated by a small interfering RNA (siRNA; see generally Fire et al., 1998), resulting in null or hypomorphic phenotypes. Thus, because described herein are nucleotide sequences encoding the stress-related proteins of the presently disclosed subject matter, RNAi can be readily designed. Indeed, constructs encoding an RNAi molecule have been developed which continuously synthesize an RNAi molecule, resulting in prolonged repression of expression of the targeted gene (Brummelkamp et al., 2002).

In transgenic plants containing AsHSP17, the expression of the nucleotide sequence corresponding to the nucleotide sequence comprised in the DNA molecule is in one embodiment reduced. In one embodiment, the nucleotide sequence in the DNA molecule is at least 70% identical to the nucleotide sequence the expression of which is reduced, in another embodiment it is at least 80% identical, in another embodiment it is at least 90% identical, in another embodiment it is at least 95% identical, and in still another embodiment it is at least 99% identical.

### 9. Insertion of a DNA Molecule (Insertional Mutagenesis)

In one embodiment, a DNA molecule is inserted into a chromosomal copy of AsHSP17, or into a regulatory region thereof. In one embodiment, such DNA molecule comprises a transposable element capable of transposition in a plant cell, such as, for example, Ac/Ds, Em/Spm, mutator. Altematively, the DNA molecule comprises a T-DNA border of an Agrobacterium T-DNA. The DNA molecule can also comprise a recombinase or integrase recognition site that can be used to remove part of the DNA molecule from the chromosome of the plant cell. Methods of insertional mutagenesis using T-DNA, transposons, oligonucleotides, or other methods known to those skilled in the art are also encompassed. Methods of using T-DNA and transposon for insertional mutagenesis are disclosed in Winkler & Feldmann, 1989, and Martienssen, 1998, incorporated herein by reference in their entireties.

### 10. Deletion Mutagenesis

In yet another embodiment, a mutation of a nucleic acid molecule of AsHSP17 can be created in the genomic copy of the sequence in the cell or plant by deletion of a portion of the nucleotide sequence or regulator sequence. Methods of deletion mutagenesis are known to those skilled in the art. See e.g., Miao & Lam, 1995.

In yet another embodiment, a deletion is created at random in a large population of plants by chemical mutagenesis or irradiation and a plant with a deletion in a gene of the presently disclosed subject matter is isolated by forward or reverse genetics. Irradiation with fast neutrons or gamma rays is known to cause deletion mutations in plants (Silverstone et al., 1998; Bruggemann et al., 1996; Redei & Koncz, 1992). Deletion mutations in a gene of AsHSP17 can be recovered in a reverse genetics strategy using PCR with pooled sets of genomic DNAs as has been shown in C. elegans (Liu et al., 1999). A forward genetics strategy involves mutagenesis of a line bearing a trait of interest followed by screening the M2 progeny for the absence of the trait. Among these mutants would be expected to be some that disrupt a gene of the presently disclosed subject matter. This could be assessed by Southem blotting or PCR using primers designed for a gene of the presently disclosed subject matter with genomic DNA from these mutants.

In yet another embodiment, AsHSP17 can be overexpressed. Examples of nucleic acid molecules and expression cassettes for over-expression of a nucleic acid molecule of the presently disclosed subject matter are disclosed herein. Methods known to those skilled in the art of over-expression of nucleic acid molecules are also encompassed by the presently disclosed subject matter.

In one embodiment, the expression of AsHSP17 can be altered in every cell of a plant. This can be obtained, for example, though homologous recombination or by insertion into a chromosome. This can also be obtained, for example, by expressing a sense or antisense RNA, zinc finger polypeptide or ribozyme under the control of a promoter capable of expressing the sense or antisense RNA, zinc finger polypeptide, or ribozyme in every cell of a plant. Constitutive, inducible, tissue-specific, cell type-specific, or developmentally-regulated expression are also within the scope of the presently disclosed subject matter and result in a constitutive, inducible, tissue-specific, or developmentally-regulated alteration of the expression of AsHSP17 in the plant cell. Constructs for expression of the sense or antisense RNA, zinc finger polypeptide, or ribozyme, or for over-expression of a nucleotide sequence of AsHSP17 can be prepared and transformed into a plant cell according to the teachings of the presently disclosed subject matter, for example, as disclosed herein.

Further encompassed within the presently disclosed subject matter is a recombinant vector comprising an expression cassette according to the embodiments of the presently disclosed subject matter. Also encompassed are plant cells comprising expression cassettes according to the present disclosure, and plants comprising these plant cells.

In one embodiment, the expression cassette is expressed throughout the plant. In another embodiment, the expression cassette is expressed in a specific location or tissue of a plant. In one embodiment, the location or tissue includes, but is not limited to, epidermis, root, vascular tissue, meristem, cambium, cortex, pith, leaf, flower, and combinations thereof. In another embodiment, the location or tissue is a seed.

In one embodiment, the expression cassette is involved in a function including, but not limited to, disease resistance, yield, biotic or abiotic stress resistance, nutritional quality, carbon metabolism, photosynthesis, signal transduction, cell growth, reproduction, disease processes (for example, pathogen resistance), gene regulation, and differentiation.

For example, a nucleic acid molecule of AsHSP17 can be introduced, under conditions for expression, into a host cell such that the host cell transcribes and translates the nucleic acid molecule to produce a stress-related polypeptide. By “under conditions for expression” is meant that a nucleic acid molecule is positioned in the cell such that it will be expressed in that cell. For example, a nucleic acid molecule can be located downstream of a promoter that is active in the cell, such that the promoter will drive the expression of the polypeptide encoded for by the nucleic acid molecule in the cell. Any regulatory sequence (e.g., promoter, enhancer, inducible promoter) can be linked to the nucleic acid molecule; alternatively, the nucleic acid molecule can include its own regulatory sequence(s) such that it will be expressed (i.e., transcribed and/or translated) in a cell.

Where the nucleic acid molecule is introduced into a cell under conditions of expression, that nucleic acid molecule can be included in an expression cassette. Thus, the presently disclosed subject matter further provides a host cell comprising an expression cassette comprising a nucleic acid molecule encoding an AsHSP17 stress-related polypeptide as disclosed herein. Such an expression cassette can include, in addition to the nucleic acid molecule encoding a stress-related polypeptide of the presently disclosed subject matter, at least one regulatory sequence (e.g., a promoter and/or an enhancer).

As such, coding sequences intended for expression in transgenic plants can be first assembled in expression cassettes operatively linked to a suitable promoter expressible in plants. The expression cassettes can also comprise any further sequences required or selected for the expression of the transgene. Such sequences include, but are not limited to, transcription terminators, extraneous sequences to enhance expression such as introns, vital sequences, and sequences intended for the targeting of the gene product to specific organelles and cell compartments. These expression cassettes can then be easily transferred to the plant transformation vectors disclosed below. The following is a description of various components of typical expression cassettes.

The selection of the promoter used in expression cassettes can determine the spatial and temporal expression pattern of the transgene in the transgenic plant. Selected promoters can express transgenes in specific cell types (such as leaf epidermal cells, mesophyll cells, root cortex cells) or in specific tissues or organs (roots, leaves, or flowers, for example) and the selection can reflect the desired location for accumulation of the gene product. Alternatively, the selected promoter can drive expression of the gene under various inducing conditions. Promoters vary in their strength; i.e., their abilities to promote transcription. Depending upon the host cell system utilized, any one of a number of suitable promoters can be used, including the gene's native promoter. The following are non-limiting examples of promoters that can be used in expression cassettes.

In one non-limiting example, a plant promoter fragment can be employed that will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various plant genes known to those of ordinary skill in the art. Such genes include for example, the AP2 gene, ACT11 from Arabidopsis (Huang et al., 1996), Cat3 from Arabidopsis (GENBANK® Accession No. U43147; Zhong et al., 1996), the gene encoding stearoyl-acyl carrier protein desaturase from Brassica napus (GENBANK® Accession No. X74782; Solocombe et al., 1994), GPc1 from maize (GENBANK© Accession No. X15596; Martinez et al., 1989), and Gpc2 from maize (GENBANK® Accession No. U45855; Manjunath et al., 1997).

Alternatively, the plant promoter can direct expression of the nucleic acid molecules of the presently disclosed subject matter in a specific tissue or can be otherwise under more precise environmental or developmental control. Examples of environmental conditions that can effect transcription by inducible promoters include anaerobic conditions, elevated temperature, or the presence of light. Such promoters are referred to herein as “inducible”, “cell type-specific”, or “tissue-specific” promoters. Ordinary skill in the art will recognize that a tissue-specific promoter can drive expression of operatively linked sequences in tissues other than the target tissue. Thus, as used herein a tissue-specific promoter is one that drives expression preferentially in the target tissue, but can also lead to some expression in other tissues as well.

Examples of promoters under developmental control include promoters that initiate transcription only (preferentially) in certain tissues, such as fruit, seeds, or flowers. Promoters that direct expression of nucleic acids in ovules, flowers, or seeds are particularly useful in the presently disclosed subject matter. As used herein a seed-specific or preferential promoter is one that directs expression specifically or preferentially in seed tissues. Such promoters can be, for example, ovule-specific, embryo-specific, endosperm-specific, integument-specific, seed coat-specific, or some combination thereof. Examples include a promoter from the ovule-specific BEL1 gene described in Reiser et al., 1995 (GENBANK© Accession No. U39944). Non-limiting examples of seed specific promoters are derived from the following genes: MAC1 from maize (Sheridan et al., 1996), Cat3 from maize (GENBANK® Accession No. L05934; Abler et al., 1993), the gene encoding oleosin 18 kD from maize (GENBANK®Accession No. J05212; Lee et al., 1994), vivparous-1 from Arabidopsis (GENBANK® Accession No. U93215), the gene encoding oleosin from Arabidopsis (GENBANK® Accession No. Z17657), Atmycl from Arabidopsis (Urao et al., 1996), the 2s seed storage protein gene family from Arabidopsis (Conceicao et al., 1994) the gene encoding oleosin 20 kD from Brassica napus (GENBANK® Accession No. M63985), napA from Brassica napus (GENBANK® Accession No. J02798; Josefsson et al., 1987), the napin gene family from Brassica napus (Sjodahl et al., 1995), the gene encoding the 2S storage protein from Brassica napus (Dasgupta et al., 1993), the genes encoding oleosin A (GENBANK® Accession No. U09118) and oleosin B (GENBANKQ Accession No. U09119) from soybean, and the gene encoding low molecular weight sulphur rich protein from soybean (Choi et al., 1995).

Alternatively, particular sequences that provide the promoter with desirable expression characteristics, or the promoter with expression enhancement activity, could be identified and these or similar sequences introduced into the sequences via cloning or via mutation. It is further contemplated that these sequences can be mutagenized in order to enhance the expression of transgenes in a particular species.

Furthermore, it is contemplated that promoters combining elements from more than one promoter can be employed. For example, U.S. Pat. No. 5,491,288 (incorporated herein by reference) discloses combining a Cauliflower Mosaic Virus (CaMV) promoter with a histone promoter. Thus, the elements from the promoters disclosed herein can be combined with elements from other promoters.

a. Constitutive Expression: the Ubiquitin Promoter

Another pattern of gene expression is root expression. A suitable root promoter is the promoter of the maize metallothionein-like (MTL) gene disclosed in de Framond, 1991, and also in U.S. Pat. No. 5,466,785, each of which is incorporated herein by reference. This “MTL” promoter is transferred to a suitable vector such as pCGN1761 ENX for the insertion of a selected gene and subsequent transfer of the entire promoter-gene-terminator cassette to a transformation vector of interest.

Wound-inducible promoters can also be suitable for gene expression. Numerous such promoters have been disclosed (e.g., Xu et al., 1993; Logemann et al., 1989; Rohrmeier & Lehle, 1993; Firek et al., 1993; Warner et al., 1993) and all are suitable for use with the presently disclosed subject matter. Logemann et al. describe the 5′ upstream sequences of the dicotyledonous potato wunl gene. Xu et al. show that a wound-inducible promoter from the dicotyledon potato (pin2) is active in the monocotyledon rice. Further, Rohrmeier & Lehle describe the cloning of the maize Wipl cDNA that is wound induced and which can be used to isolate the cognate promoter using standard techniques. Similarly, Firek et al. and Warner et al. have disclosed a wound-induced gene from the monocotyledon Asparagus officinalis, which is expressed at local wound and pathogen invasion sites. Using cloning techniques well known in the art, these promoters can be transferred to suitable vectors, fused to the genes pertaining to the presently disclosed subject matter, and used to express these genes at the sites of plant wounding.

PCT International Publication WO 93/07278, which is herein incorporated by reference, describes the isolation of the maize trpA gene, which is preferentially expressed in pith cells. The gene sequence and promoter extending up to −1726 basepairs (bp) from the start of transcription are presented. Using standard molecular biological techniques, this promoter, or parts thereof, can be transferred to a vector such as pCGN1761 where it can replace the 35S promoter and be used to drive the expression of a foreign gene in a pith-preferred manner. In fact, fragments containing the pith-preferred promoter or parts thereof can be transferred to any vector and modified for utility in transgenic plants.

A maize gene encoding phosphoenol carboxylase (PEPC) has been disclosed by Hudspeth & Grula, 1989. Using standard molecular biological techniques, the promoter for this gene can be used to drive the expression of any gene in a leaf-specific manner in transgenic plants.

WO 93/07278 (incorporated herein by reference) describes the isolation of the maize calcium-dependent protein kinase (CDPK) gene that is expressed in pollen cells. The gene sequence and promoter extend up to 1400 bp from the start of transcription. Using standard molecular biological techniques, this promoter or parts thereof can be transferred to a vector such as pCGN1761 where it can replace the 35S promoter and be used to drive the expression of a nucleic acid sequence of the presently disclosed subject matter in a pollen-specific manner.

A variety of 5′ and 3′ transcriptional regulatory sequences are available for use in the presently disclosed subject matter. Transcriptional terminators are responsible for the termination of transcription and correct mRNA polyadenylation. The 3′ nontranslated regulatory DNA sequence includes from in one embodiment about 50 to about 1,000, and in another embodiment about 100 to about 1,000, nucleotide base pairs and contains plant transcriptional and translational termination sequences. Appropriate transcriptional terminators and those that are known to function in plants include the CaMV 35S terminator, the tml terminator, the nopaline synthase terminator, the pea rbcS E9 terminator, the terminator for the T7 transcript from the octopine synthase gene of Agrobacterium tumefaciens, and the 3′ end of the protease inhibitor I or II genes from potato or tomato, although other 3′ elements known to those of skill in the art can also be employed. Alternatively, a gamma coixin, oleosin 3, or other terminator from the genus Coix can be used.

Non-limiting 3′ elements include those from the nopaline synthase gene of Agrobacterium tumefaciens (Bevan et al., 1983), the terminator for the T7 transcript from the octopine synthase gene of Agrobacterium tumefaciens, and the 3′ end of the protease inhibitor I or II genes from potato or tomato.

As the DNA sequence between the transcription initiation site and the start of the coding sequence (i.e., the untranslated leader sequence, also referred to as the 5′ untranslated region) can influence gene expression, a particular leader sequence can also be employed. Non-limiting leader sequences are contemplated to include those that include sequences predicted to direct optimum expression of the operatively linked gene; i.e., to include a consensus leader sequence that can increase or maintain mRNA stability and prevent inappropriate initiation of translation. The choice of such sequences will be known to those of skill in the art in light of the present disclosure. Sequences that are derived from genes that are highly expressed in plants are useful in the presently disclosed subject matter.

Thus, a variety of transcriptional terminators are available for use in expression cassettes. These are responsible for termination of transcription and correct mRNA polyadenylation. Appropriate transcriptional terminators are those that are known to function in plants and include the CaMV 35S terminator, the tml terminator, the nopaline synthase terminator, and the pea rbcS E9 terminator. These can be used in both monocotyledons and dicotyledons. In addition, a gene's native transcription terminator can be used.

Numerous sequences have been found to enhance gene expression from within the transcriptional unit and these sequences can be used in conjunction with the AsHSP17 sequences to increase their expression in transgenic plants.

Other sequences that have been found to enhance gene expression in transgenic plants include intron sequences (e.g., from Adhl, bronze1, actin1, actin 2 (PCT International Publication No. WO 00/760067 (incorporated herein by reference)), or the sucrose synthase intron), and viral leader sequences (e.g., from Tobacco Mosaic Virus (TMV), Maize Chlorotic Mottle Virus (MCMV), or Alfalfa Mosaic Virus (AMV)). For example, a number of non-translated leader sequences derived from viruses are known to enhance the expression of operatively linked nucleic acids. Specifically, leader sequences from Tobacco Mosaic Virus (TMV), Maize Chlorotic Mottle Virus (MCMV), and Alfalfa Mosaic Virus (AMV) have been shown to be effective in enhancing expression (e.g., Gallie et al., 1987; Skuzeski et al., 1990). Other leaders known in the art include, but are not limited to picomavirus leaders, for example, encephalomyocarditis virus (EMCV) leader (encephalomyocarditis 5′ noncoding region; Elroy-Stein et al., 1989); potyvirus leaders (e.g., Tobacco Etch Virus (TEV) leader and Maize Dwarf Mosaic Virus (MDMV) leader); human immunoglobulin heavy-chain binding protein (BiP) leader (Macejak et al., 1991); untranslated leader from the coat protein mRNA of AMV (AMV RNA 4; Jobling et al., 1987); TMV leader (Gallie et al., 1989); and maize chlorotic mottle virus leader (Lommel et al., 1991). See also, Della-Cioppa et al., 1987. Regulatory elements such as Adh intron 1 (Callis et al., 1987), sucrose synthase intron (Vasil et al., 1989) or TMV omega element (Gallie et al., 1989), can further be included where desired. Non-limiting examples of enhancers include elements from the CaMV 35S promoter, octopine synthase genes (Ellis et al., 1987), the rice actin I gene, the maize alcohol dehydrogenase gene (Callis et al., 1987), the maize shrunken I gene (Vasil et al., 1989), TMV omega element (Gallie et al., 1989) and promoters from non-plant eukaryotes (e.g., yeast; Ma et al., 1988).

A number of non-translated leader sequences derived from viruses are also known to enhance expression, and these are particularly effective in dicotyledonous cells. Specifically, leader sequences from Tobacco Mosaic Virus (TMV; the “W-sequence”), Maize Chlorotic Mottle Virus (MCMV), and Alfalfa Mosaic Virus (AMV) have been shown to be effective in enhancing expression (see e.g., Gallie et al., 1987; Skuzeski et al., 1990). Other leader sequences known in the art include, but are not limited to, picomavirus leaders, for example, EMCV (encephalomyocarditis virus) leader (5′ noncoding region; see Elroy-Stein et al., 1989); potyvirus leaders, for example, from Tobacco Etch Virus (TEV; see Allison et al., 1986); Maize Dwarf Mosaic Virus (MDMV; see Kong & Steinbiss 1998); human immunoglobulin heavy-chain binding polypeptide (BiP) leader (Macejak & Samow, 1991); untranslated leader from the coat polypeptide mRNA of alfalfa mosaic virus (AMV; RNA 4; see Jobling & Gehrke, 1987); tobacco mosaic virus (TMV) leader (Gallie et al., 1989); and Maize Chlorotic Mottle Virus (MCMV) leader (Lommel et al., 1991). See also, Della-Cioppa et al., 1987.

In addition to incorporating one or more of the aforementioned elements into the 5′ regulatory region of a target expression cassette of the presently disclosed subject matter, other elements can also be incorporated. Such elements include, but are not limited to, a minimal promoter. By minimal promoter it is intended that the basal promoter elements are inactive or nearly so in the absence of upstream or downstream activation. Such a promoter has low background activity in plants when there is no transactivator present or when enhancer or response element binding sites are absent. One minimal promoter that is particularly useful for target genes in plants is the Bz1 minimal promoter, which is obtained from the bronze1 gene of maize. The Bz1 core promoter is obtained from the “myc” mutant Bz1-luciferase construct pBz1LucR98 via cleavage at the NheI site located at positions −53 to −58 (Roth et al., 1991). The derived Bz1 core promoter fragment thus extends from positions −53 to +227 and includes the Bz1 intron-1 in the 5′ untranslated region. Also useful for the presently disclosed subject matter is a minimal promoter created by use of a synthetic TATA element. The TATA element allows recognition of the promoter by RNA polymerase factors and confers a basal level of gene expression in the absence of activation (see generally, Mukumoto et al., 1993; Green, 2000.

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various polypeptides that is cleaved during chloroplast import to yield the mature polypeptides (see e.g., Comai et al., 1988). These signal sequences can be fused to heterologous gene products to affect the import of heterologous products into the chloroplast (Van den Broeck et al., 1985). DNA encoding for appropriate signal sequences can be isolated from the 5′ end of the cDNAs encoding the ribulose-1,5-bisphosphate carboxylase/oxygenase (RUBISCO) polypeptide, the chlorophyll a/b binding (CAB) polypeptide, the 5-enol-pyruvyl shikimate-3-phosphate (EPSP) synthase enzyme, the GS2 polypeptide and many other polypeptides which are known to be chloroplast localized. See also, the section entitled “Expression With Chloroplast Targeting” in Example 37 of U.S. Pat. No. 5,639,949, herein incorporated by reference.

Other gene products can be localized to other organelles such as the mitochondrion and the peroxisome (e.g., Unger et al., 1989). The cDNAs encoding these products can also be manipulated to effect the targeting of heterologous gene products to these organelles. Examples of such sequences are the nuclear-encoded ATPases and specific aspartate amino transferase isoforms for mitochondria. Targeting cellular polypeptide bodies has been disclosed by Rogers et al., 1985.

In addition, sequences have been characterized that control the targeting of gene products to other cell compartments. Amino terminal sequences are responsible for targeting to the endoplasmic reticulum (ER), the apoplast, and extracellular secretion from aleurone cells (Koehler & Ho, 1990). Additionally, amino terminal sequences in conjunction with carboxy terminal sequences are responsible for vacuolar targeting of gene products (Shinshi et al., 1990).

By the fusion of the appropriate targeting sequences disclosed above to transgene sequences of interest it is possible to direct the transgene product to any organelle or cell compartment. For chloroplast targeting, for example, the chloroplast signal sequence from the RUBISCO gene, the CAB gene, the EPSP synthase gene, or the GS2 gene is fused in frame to the amino terminal ATG of the transgene. The signal sequence selected can include the known cleavage site, and the fusion constructed can take into account any amino acids after the cleavage site that are required for cleavage. In some cases this requirement can be fulfilled by the addition of a small number of amino acids between the cleavage site and the transgene ATG or, alternatively, replacement of some amino acids within the transgene sequence. Fusions constructed for chloroplast import can be tested for efficacy of chloroplast uptake by in vitro translation of in vitro transcribed constructions followed by in vitro chloroplast uptake using techniques disclosed by Bartlett et al., 1982 and Wasmann et al., 1986. These construction techniques are well known in the art and are equally applicable to mitochondria and peroxisomes.

The above-disclosed mechanisms for cellular targeting can be utilized not only in conjunction with their cognate promoters, but also in conjunction with heterologous promoters so as to effect a specific cell-targeting goal under the transcriptional regulation of a promoter that has an expression pattern different from that of the promoter from which the targeting signal derives.

Once an AsHSP17 nucleic acid construct has been cloned into an expression system, it can be transformed into a plant cell. The receptor and target expression cassettes of the presently disclosed subject matter can be introduced into the plant cell in a number of art-recognized ways. Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Transformation of a plant can be undertaken with a single DNA molecule or multiple DNA molecules (i.e., co-transformation), and both these techniques are suitable for use with the expression cassettes of the presently disclosed subject matter. Numerous transformation vectors are available for plant transformation, and the expression cassettes of the presently disclosed subject matter can be used in conjunction with any such vectors. The selection of vector will depend upon the transformation technique and the species targeted for transformation.

A variety of techniques are available and known for introduction of nucleic acid molecules and expression cassettes comprising such nucleic acid molecules into a plant cell host. These techniques include, but are not limited to transformation with DNA employing A. tumefaciens or A. rhizogenes as the transforming agent, liposomes, PEG precipitation, electroporation, DNA injection, direct DNA uptake, microprojectile bombardment, particle acceleration, and the like (see e.g., EP 0 295 959 and EP 0 138 341).

Expression vectors containing genomic or synthetic fragments can be introduced into protoplasts or into intact tissues or isolated cells. In some embodiments, expression vectors are introduced into intact tissue. “Plant tissue” includes differentiated and undifferentiated tissues or entire plants, including but not limited to roots, stems, shoots, leaves, pollen, seeds, tumor tissue, and various forms of cells and cultures such as single cells, protoplasts, embryos, and callus tissues. The plant tissue can be in plants or in organ, tissue, or cell culture. General methods of culturing plant tissues are provided, for example, by Maki et al., 1993 and by Phillips et al. 1988. In some embodiments, expression vectors are introduced using a direct gene transfer method such as microprojectile-mediated delivery, DNA injection, electroporation, or the like. In some embodiments, expression vectors are introduced into plant tissues using microprojectile media delivery with a biolistic device (see e.g., Tomes et al., 1995). The vectors can not only be used for expression of structural genes but can also be used in exon-trap cloning or in promoter trap procedures to detect differential gene expression in varieties of tissues (Lindsey et al., 1993; Auch & Reth, 1990).

In some embodiments, the binary type vectors of the Ti and Ri plasmids of Agrobacterium spp are employed. Ti-derived vectors can be used to transform a wide variety of higher plants, including monocotyledonous and dicotyledonous plants including, but not limited to soybean, cotton, rape, tobacco, and rice (Pacciotti et al., 1985: Byre et al., 1987; Sukhapinda et al., 1987; Lorz et al., 1985; Potrykus, 1985; Park et al., 1985: Hiei et al., 1994). The use of T-DNA to transform plant cells has received extensive study and is amply described (European Patent Application No. EP 0 120 516; Hoekema, 1985; Knauf et al., 1983; and An et al., 1985, each of which is incorporated by reference in its entirety).

Other transformation methods are available to those skilled in the art, such as direct uptake of foreign DNA constructs (see European Patent Application No. EP 0 295 959), electroporation (Fromm et al., 1986), or high velocity ballistic bombardment of plant cells with metal particles coated with the nucleic acid constructs (Kline et al., 1987; U.S. Pat. No. 4,945,050). Once transformed, the cells can be regenerated using techniques familiar to those of skill in the art. Of particular relevance are the recently described methods to transform foreign genes into commercially important crops, such as rapeseed (De Block et al., 1989), sunflower (Everett et al., 1987), soybean (McCabe et al., 1988; Hinchee et al., 1988; Chee et al., 1989; Christou et al., 1989; European Patent Application No. EP 0 301 749), rice (Hiei et al., 1994), and corn (Gordon Kamm et al., 1990; Fromm et al., 1990).

Of course, the choice of method might depend on the type of plant targeted for transformation. Suitable methods of transforming plant cells include, but are not limited to microinjection (Crossway et al., 1986), electroporation (Riggs et al., 1986), Agrobacterium-mediated transformation (Hinchee et al., 1988), direct gene transfer (Paszkowski et al., 1984), and ballistic particle acceleration using devices available from Agracetus, Inc. (Madison, Wis., United States of America) and BioRad (Hercules, Calif., United States of America). See e.g., U.S. Pat. No. 4,945,050; McCabe et al., 1988; Weissinger et al., 1988; Sanford et al., 1987 (onion); Christou et al., 1988 (soybean); McCabe et al., 1988 (soybean); Datta et al., 1990 (rice); Klein et al., 1988 (maize); Fromm et al., 1990 (maize); Gordon-Kamm et al., 1990 (maize); Svab et al., 1990 (tobacco chloroplast); Koziel et al., 1993 (maize); Shimamoto et al., 1989 (rice); Christou et al., 1991 (rice); European Patent Application EP 0 332 581 (orchardgrass and other Pooideae); Vasil et al., 1993 (wheat); Weeks et al., 1993 (wheat). In one embodiment, the protoplast transformation method for maize is employed (see European Patent Application EP 0 292 435; U.S. Pat. No. 5,350,689).

Agrobacterium tumefaciens cells containing a vector comprising an expression cassette of the presently disclosed subject matter, wherein the vector comprises a Ti plasmid, are useful in methods of making transformed plants. Plant cells are infected with an Agrobacterium tumefaciens to produce a transformed plant cell, and then a plant is regenerated from the transformed plant cell. Numerous Agrobacterium vector systems useful in carrying out the presently disclosed subject matter are known to ordinary skill in the art.

Many vectors are available for transformation using Agrobacterium tumefaciens. These typically carry at least one T-DNA border sequence and include vectors such as pBIN19 (Bevan, 1984). For instance, the binary vectors pCIB200 and pCIB2001 can be used for the construction of recombinant vectors for use with Agrobacterium and can be constructed according to known methodology.

Transformation without the use of Agrobacterium tumefaciens circumvents the requirement for T-DNA sequences in the chosen transformation vector, and consequently vectors lacking these sequences can be utilized in addition to vectors such as the ones disclosed above that contain T-DNA sequences. Transformation techniques that do not rely on Agrobacterium include transformation via particle bombardment, protoplast uptake (e.g., polyethylene glycol (PEG) and electroporation), and microinjection. The choice of vector depends largely on the species being transformed.

Methods using either a form of direct gene transfer or Agrobacterium-mediated transfer usually, but not necessarily, are undertaken with a selectable marker that can provide resistance to an antibiotic (e.g., kanamycin, hygromycin, or methotrexate) or a herbicide (e.g., phosphinothricin). The choice of selectable marker for plant transformation is not, however, critical to the presently disclosed subject matter.

For certain plant species, different antibiotic or herbicide selection markers can be employed. Selection markers used routinely in transformation include the nptII gene, which confers resistance to kanamycin and related antibiotics (Messing & Vierra, 1982; Bevan et al., 1983), the bar gene, which confers resistance to the herbicide phosphinothricin (White et al., 1990, Spencer et al., 1990), the hph gene, which confers resistance to the antibiotic hygromycin (Blochinger & Diggelmann, 1984), and the dhfr gene, which confers resistance to methotrexate (Bourouis et al., 1983).

Selection markers resulting in positive selection, such as a phosphomannose isomerase (PMI) gene (described in PCT International Publication No. WO 93/05163) can also be used. Other genes that can be used for positive selection are described in PCT International Publication No. WO 94/20627 and encode xyloisomerases and phosphomanno-isomerases such as mannose-6-phosphate isomerase and mannose-1-phosphate isomerase; phosphomanno mutase; mannose epimerases such as those that convert carbohydrates to mannose or mannose to carbohydrates such as glucose or galactose; phosphatases such as mannose or xylose phosphatase, mannose-6-phosphatase and mannose-1-phosphatase, and permeases that are involved in the transport of mannose, or a derivative or a precursor thereof, into the cell. An agent is typically used to reduce the toxicity of the compound to the cells, and is typically a glucose derivative such as methyl-3-O-glucose or phloridzin. Transformed cells are identified without damaging or killing the non-transformed cells in the population and without co-introduction of antibiotic or herbicide resistance genes. As described in PCT International Publication No. WO 93/05163, in addition to the fact that the need for antibiotic or herbicide resistance genes is eliminated, it has been shown that the positive selection method is often far more efficient than traditional negative selection.

For expression of a nucleotide sequence of the presently disclosed subject matter in plant plastids, plastid transformation vector pPH143 (PCT Intemational Publication WO 97/32011, example 36) can be used. The nucleotide sequence is inserted into pPH143 thereby replacing the protoporphyrinogen oxidase (Protox) coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

In another embodiment, a nucleotide sequence of the presently disclosed subject matter is directly transformed into the plastid genome. Plastid transformation technology is described in U.S. Pat. Nos. 5,451,513; 5,545,817; and 5,545,818; and in PCT International Publication No. WO 95/16783; and in McBride et al., 1994.

Another approach to transforming plant cells involves propelling inert or biologically active particles at plant tissues and cells. This technique is disclosed in U.S. Pat. Nos. 4,945,050; 5,036,006; and 5,100,792; all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium, or a bacteriophage, each containing DNA sought to be introduced) can also be propelled into plant cell tissue.

Transformation of most monocotyledon can include direct gene transfer into protoplasts using PEG or electroporation, and particle bombardment into callus tissue. Transformations can be undertaken with a single DNA species or multiple DNA species (i.e. co-transformation), and both these techniques are suitable for use with the presently disclosed subject matter. Co-transformation can have the advantage of avoiding complete vector construction and of generating transgenic plants with unlinked loci for the gene of interest and the selectable marker, enabling the removal of the selectable marker in subsequent generations, should this be regarded as desirable. However, a disadvantage of the use of co-transformation is the less than 100% frequency with which separate DNA species are integrated into the genome (Schocher et al., 1986).

Transformation of monocotyledons using Agrobacterium has also been disclosed. See WO 94/00977 and U.S. Pat. No. 5,591,616, both of which are incorporated herein by reference. See also Negrotto et al., 2000, Zhao et al., 2000, and also U.S. Pat. No. 6,369,298, which is incorporated herein by reference.

Once formed, transgenic plant cells can be placed in an appropriate selective medium for selection of transgenic cells, which are then grown to callus. Shoots are grown from callus and plantlets generated from the shoot by growing in rooting medium. The various constructs normally are joined to a marker for selection in plant cells. Conveniently, the marker can be resistance to a biocide (for example, an antibiotic including, but not limited to kanamycin, G418, bleomycin, hygromycin, chloramphenicol, herbicide, or the like). The particular marker used is designed to allow for the selection of transformed cells (as compared to cells lacking the DNA that has been introduced). Components of DNA constructs including transcription cassettes of the presently disclosed subject matter are prepared from sequences that are native (endogenous) or foreign (exogenous) to the host. As used herein, the terms “foreign” and “exogenous” refer to sequences that are not found in the wild-type host into which the construct is introduced, or alternatively, have been isolated from the host species and incorporated into an expression vector. Heterologous constructs contain in one embodiment at least one region that is not native to the gene from which the transcription initiation region is derived.

To confirm the presence of the transgenes in transformed cells and plants, a variety of assays can be performed. Such assays include, for example, “molecular biological” assays well known to those of skill in the art, such as Southern and Northern blotting, in situ hybridization and nucleic acid-based amplification methods such as PCR or RT-PCR; “biochemical” assays, such as detecting the presence of a protein product, e.g., by immunological means (enzyme-linked immunosorbent assays (ELISAs) and Western blots) or by enzymatic function; plant part assays, such as seed assays; and also by analyzing the phenotype of the whole regenerated plant, e.g., for disease or pest resistance.

DNA can be isolated from cell lines or any plant parts to determine the presence of the preselected nucleic acid segment through the use of techniques well known to those skilled in the art. Note that intact sequences will not always be present, presumably due to rearrangement or deletion of sequences in the cell.

The present disclosure may be better understood with reference to the Example, set forth below.

### Example

### Experimental Procedures

### Plant Materials

A commercial creeping bentgrass (A. stolonifera L.) cultivar, Penn A-4 provided by HybriGene (Hubbard, Oreg.) was grown under standard growth room condition with a 16 h photoperiod with supplemental lighting at 27° C. in the light and 25° C. in the dark. The plants were clonally propagated from stolons and grown in small containers (4.0×20.3 cm, Dillen Products, Middlefield, Ohio, USA) using pure silica sand. After three weeks of maintenance in the growth room, the plants were treated at 37° C. for heat stress; 200 mM NaCl for salt stress; water withhold for drought stress and 50 μM ABA for 4 h respectively. The first leaf of each stolon collected at 0 h, 0.5 h, 2 h, 4 h and root collected at 0 h and 4 h of treatment were used for RNA isolation to clone the AsHSP17 gene and to test AsHSP17 gene expression level.

Arabidopsis thaliana (ecotype Columbia) plants used for genetic transformation was grown in a growth chamber (Conviron, Controlled Environments Inc., Pembina, N. Dak.) at 23/20° C. with 16/8 h (light/dark) photoperiod.

### Plant Genomic DNA, RNA Isolation and Semi-Quantitative RT-PCR

Plant genomic DNA was isolated by cetyltrimethyl ammonium bromide method as described by Luo et al. (2005). Total RNA was extracted with Trizol reagent (Invitrogen, Carlsbad, Calif.) and used for cDNA synthesis using Superscript III System (Invitrogen). The cDNA were used for semi-quantitative PCR with gene-specific primers. Creeping bentgrass ubiquitin gene were used as a reference gene.

### Plasmid Construction and Plant Transformation

The AsHSP17 chimeric gene overexpression construct, p35S-AsHSP17/35S-bar which contains two cauliflower mosaic virus 35S (CaMV35S) promoters driving the turfgrass heat shock protein 17 gene AsHSP17 and bar gene for herbicide resistance respectively. The construct was transformed into Agrobaterium tumefaciens LB4404 strain and introduced into wild type Arabidopsis thaliana (Col-0) using floral dip. Individual transgenic plants were selected on the basis of resistance to herbicide treatment.

### Subcellular Localization of AsHSP17

The full-length of AsHSP17 coding region without stop codon was subcloned into p35S-AsHSP17-sGFP/35S-bar vector with the GFP at C-terminus and the identity of the plasmid construct was confirmed by sequencing. The resulting fusion construct and the control construct (contained GFP only) were transformed into the Arabidopsis. The mesophyll protoplast isolated from 3 weeks old transgenic plant leaves were used for fluorescence observation by confocal scanning microscopy (Nikon) Arabidopsis mesophyll protoplast isolated according to Yoo's methods (Yoo et al. 2007)

### AsHSP17 Promoter Construction and GUS Staining

A 3-kb sequence upstream of the AsHSP17 start codon which obtained by using inverse PCR strategy were amplified and subcloned into pAsHSP17-GUS/35S-bar binary vector. The construct was transformed into Arabidopsis by using floral dip. The transgenic plants under different stress samples were harvested and incubated in staining buffer (50 mM sodium phosphate buffer, pH 7.2, 0.2% Triton-X, 2 mM potassium ferrocyanide, 2 mM potassium ferrcyanide, 2 mM 5-bromo-4-chloro-3-indolyl-β-D-glucuronide cyclohexamine salt-dissolved in N,N, dimethy formamide) at 37° C. overnight. Samples were then washed in 70% ethanol before taking images.

### Heat Stress Treatment

Seeds of four events of transgenic (TG1, TG2, TG4 and TG5) and WT control plants were sown directly on the 3-B Mix potting soil (Fafard Inc., Anderson, S.C., USA) and kept at 4° C. for 3d before proceeding to stress-related experiments in a growth chamber.

For heat stress, 3 weeks old plants were transferred to high temperature condition in a growth chamber set to 40° C./40° C. day/night with 13/11 h (light/dark) photoperiod. The relative humidity in the chamber was 60%-80% and the heat-stressed plants were well-watered.

### Seed Germination Assays

Seed germination assays under salt stress condition were performed by placing WT and TG seeds on % MS media containing 125 mM, 150 mM and 175 mM NaCl, and the numbers of germinated seeds and green seedlings were counted on the fourth day. Germination was defined as the complete protrusion of the radicle. Greening was defined as the cotyledons were fully expanded.

Seed germination assays under ABA condition were performed by placing WT and TG seeds on % MS media containing 0.75 μM and 1 μM ABA, and the numbers of germinated seeds and green seedlings were counted on the fourth day and sixth day.

### Leaf Electrolyte Leakage, Chlorophyll, Relative Water Contents

Leaf electrolyte leakage (EL), chlorophyll, relative water contents (RWC) were measured according to standard methodology.

### Statistical Analysis

The significance of differences between data sets was evaluated by Student's t-test. Asterisks (*, ** or ***) indicate a significant difference between TG plants and WT controls at P<0.05, 0.01 or 0.001, respectively.

Isolation and Sequence Analysis of HSP17 Cloned from Creeping Bentgrass

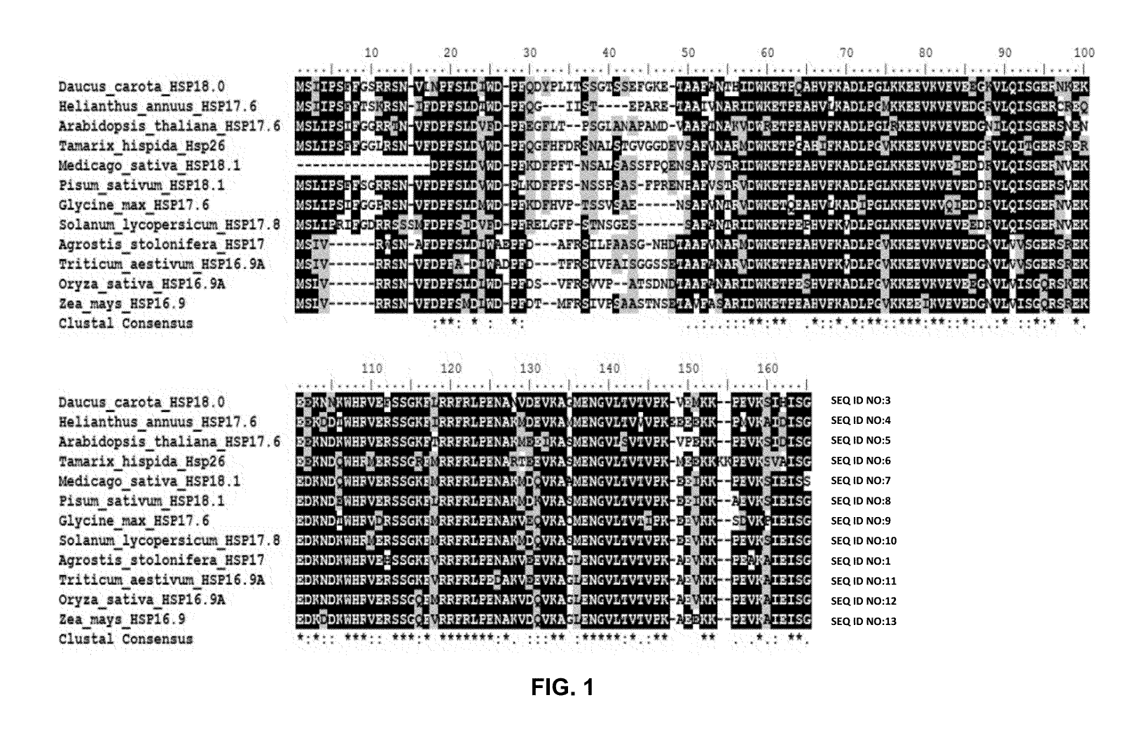

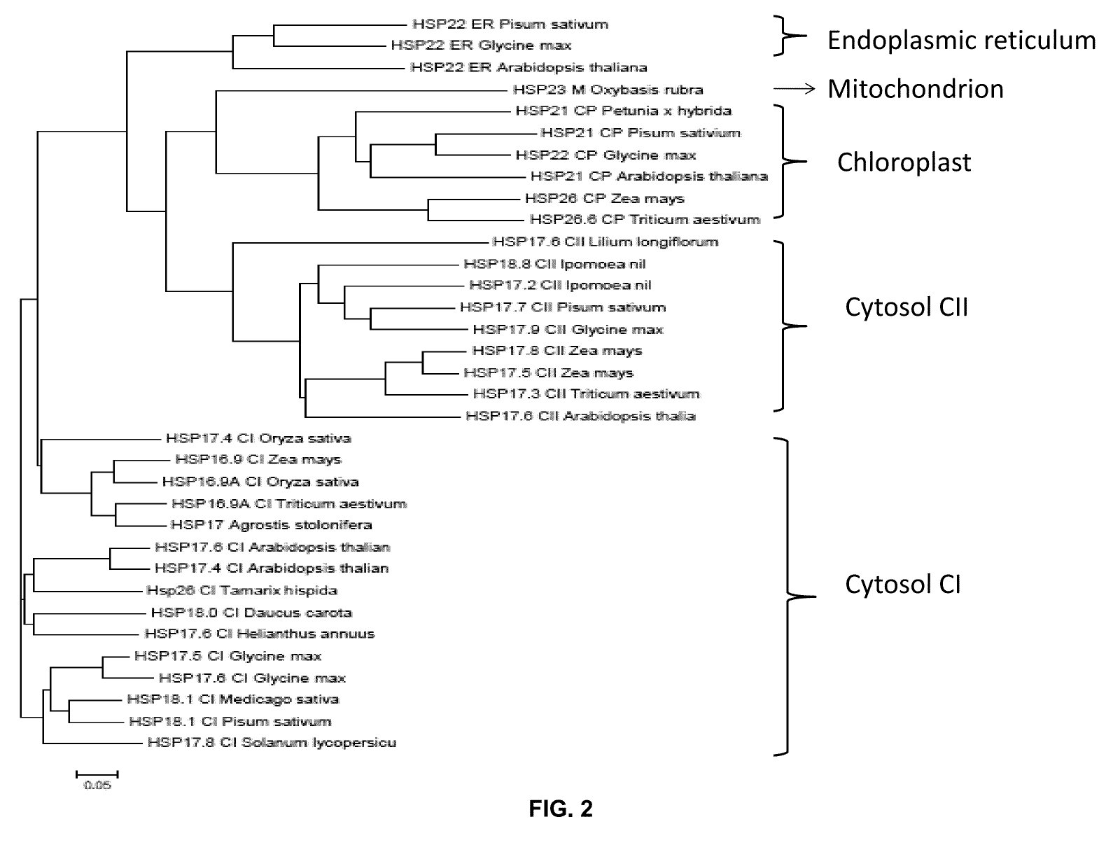

To study the potential of manipulating the small heat shock protein AsHSP17 expression in plant for impact plant resistance to abiotic stress, we cloned the AsHSP17 from creeping bentgrass (Agrostis stolonifera L.) cultivar, Penn A-4. The length of AsHSP17 open reading frame (ORF) is 456 bp (SEQ ID NO: 2), which encodes a peptide of 151 amino acids (SEQ ID NO: 1). Alignment of the amino acid sequences of AsHSP17 and other representative cytosolic calss I sHSPs from other plants shows that the AsHSP17 protein contains an α-crystallin domain which including 2 sHSPs conserved domains (FIG. 1). Phylogenetic analysis of AsHSP17 with other different classes sHSPs shows that AsHSP17 is closed to cytosolic class I sHSPs (FIG. 2).

### AsHSP17 Subcellular Localization

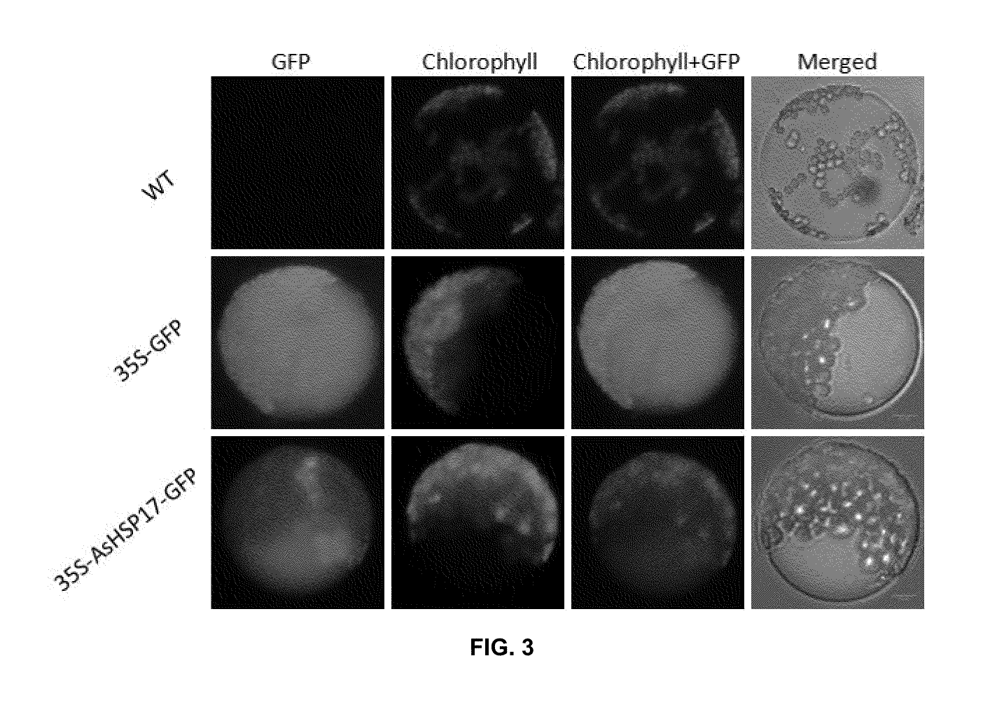

To confirm that AsHSP17 belongs to cytosolic sHSP family, the amino acid sequence of AsHSP17 was analyzed on ProtComp v.9.0 database. AsHSP17 was predicted to be localized to cytoplasm with score 9.6. In order to further confirm this prediction, AsHSP17-green fluorescent protein (GFP) fusion was introduced into Arabidopsis thaliana by using floral dip. The mesophyll protoplasts of transgenic 3 weeks old plant were prepared for fluorescent observation. AsHSP17 was found to be localized to cytoplasm (FIG. 3).

### AsHSP17 Gene Expression Patterns

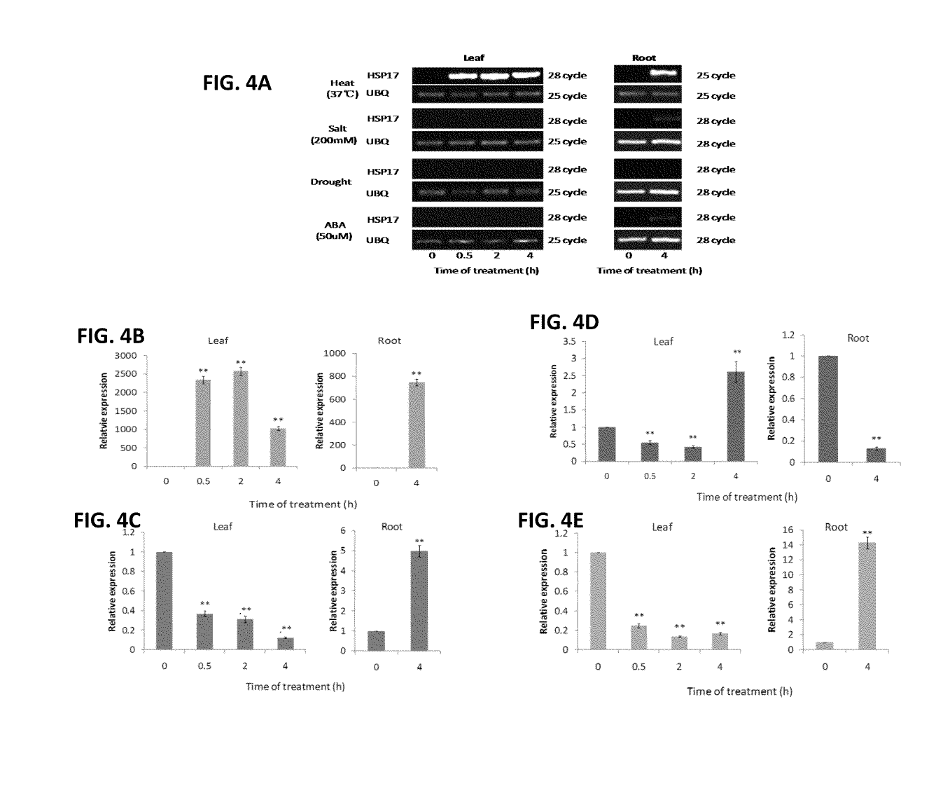

The expression profile of the AsHSP17 gene was analyzed by RT-PCR on cDNA extracted from leaf (first leaf of each stolon) at 0, 0.5, 2, 4 h after heat, salt, drought or ABA stress treatment and root at 0, 4 h after each treatment. FIG. 4A-4E illustrates the AsHSP17 expression profile in leaf (left) and root (right) under heat (37° C.), salt(200 mM NaCl), Drought, ABA(50 uM) treatment. Leaf samples were collected at 0, 0.5, 2, 4 hr after stress treatment and root samples were collected at 0,4 hr after stress treatment. The ΔΔCt method was used for real time PCR analysis. Creeping bentgrass ubiquitin gene were used as the endogenous control. “*” or “**” indicate significant differences between 0 h of treatment and other time of treatment at P<0.05 or 0.01, respectively. Error bars indicate SE (n=6).

As shown in FIG. 4A and FIG. 4B, no expression of AsHSP17 was detected before stress treatment in both leaf and root. In leaves, 0.5 h of heat stress strongly induced the expression of AsHSP17 (FIG. 4A, FIG. 4B). However, no expression of AsHSP17 was detected in leaf under salinity (FIG. 4C), drought (FIG. 4D) and ABA treatment (FIG. 4E). In root, no expression of AsHSP17 was observed under drought condition (FIG. 4D). Heat stress also strongly induced the accumulation of AsHSP17 transcripts (FIG. 4B), whereas salinity (FIG. 4C) and ABA (FIG. 4D) slightly increased the expression of AsHSP17. These results indicated that AsHSP17 might be involved in plant response to abiotic stress in creeping bentgrass.

Production and Molecular Characterization of Transgenic Arabidopsis thaliana Plants Expressing the Creeping Bentgrass AsHSP17 Gene

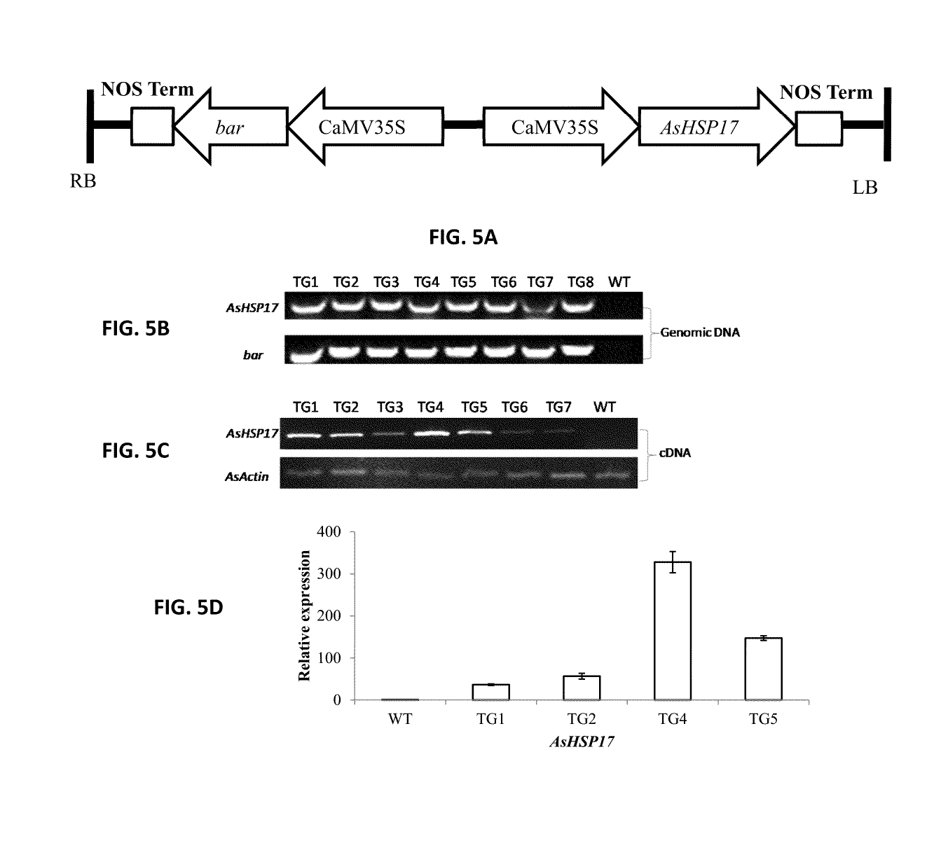

FIG. 5A-FIG. 5D presents the generation and molecular analysis of AsHSP17 in transgenic (TG) Arabidopsis thaliana. The chimeric DNA construct, p35S-AsHSP17/35S-bar, containing AsHSP17, under control of the cauliflower mosaic virus (CaMV) 35S promoter (FIG. 5A) was introduced into Arabidopsis thaliana ecotype Columbia to produce a total of 8 independent transgenic lines. PCR analysis of genomic DNA from all transformants revealed the insertion of transgenes (FIG. 5B). RT-PCR analysis demonstrated that the expression of AsHSP17 in TG1 toTG7 (FIG. 5C, FIG. 5D), which were all morphologically similar to wild type controls. TG1, TG2, TG4, TG5 were selected for further analysis (FIG. 5C, FIG. 5D).

### Overexpressing AsHSP17 Transgenic Plants Under Heat Stress

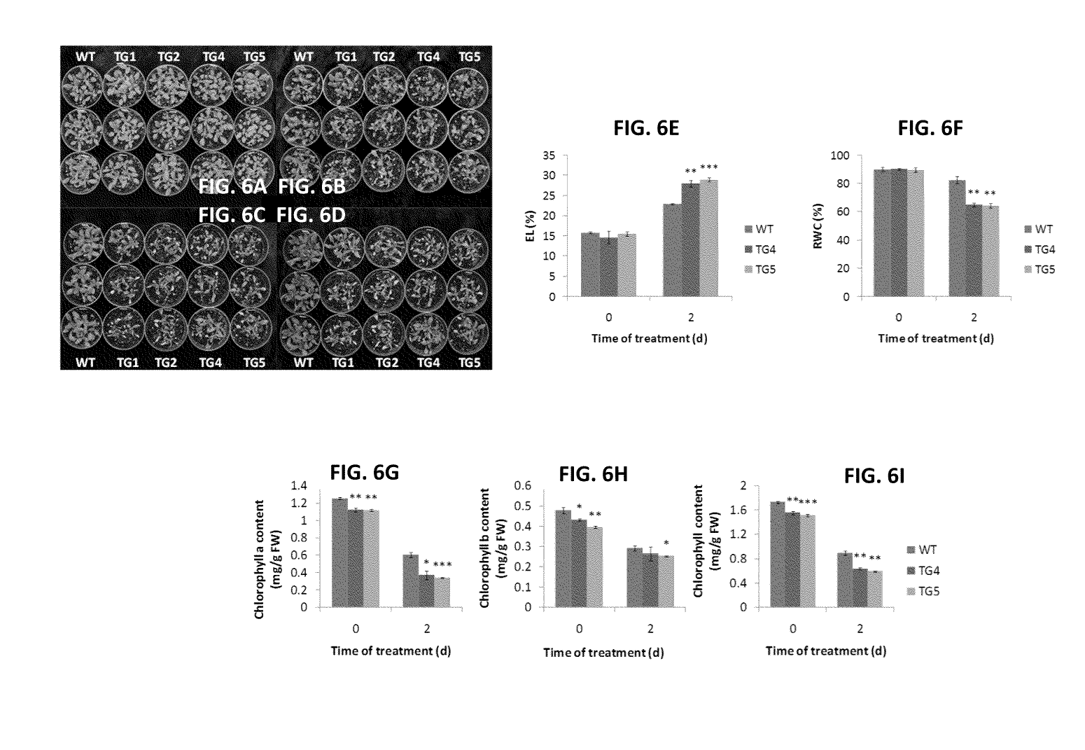

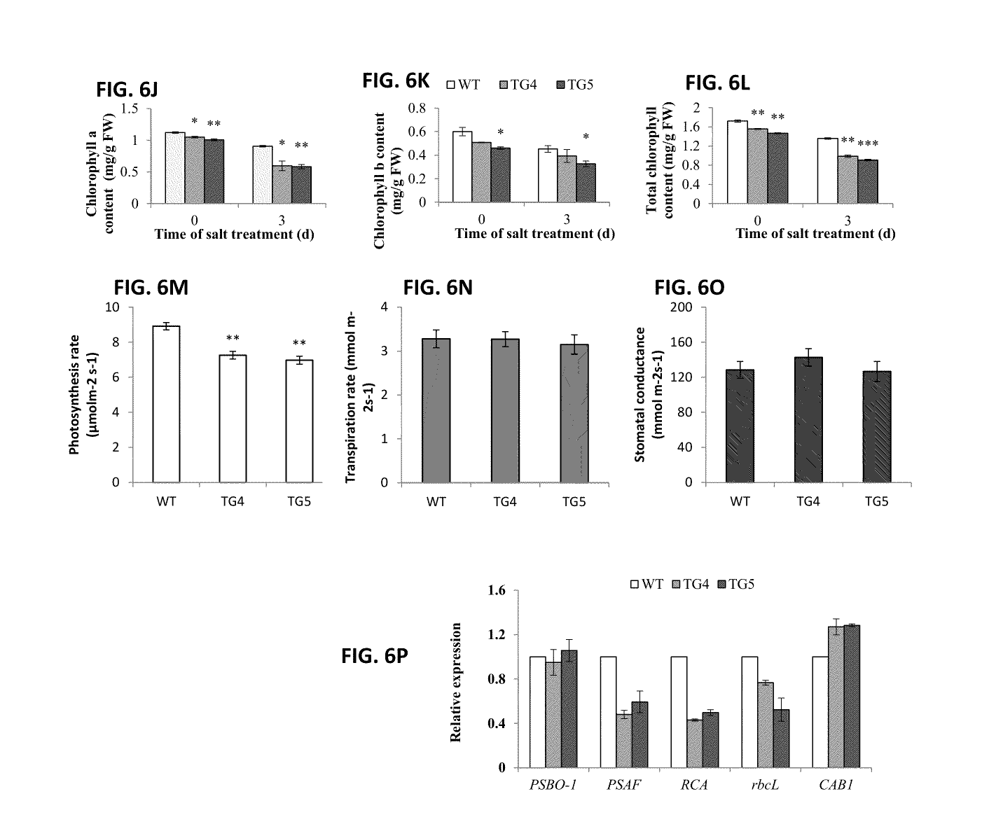

To investigate how modified expression of AsHSP17 would impact plant response to heat stress, performance of the AsHSP17-overexpressing TG lines grown under heat stress (40° C.) were examined. As shown in FIG. 6A, FIG. 6B, FIG. 6C, and FIG. 6D, after 2 days of heat treatment, TG plants became largely wilted while WT plants only exhibited minor heat damage (FIG. 6B). After 4 days of recovery, all the WT plants recovered from the heat-elicited damage and survived the treatment, whereas most of the developed leaves of TG plants died from heat stress (FIG. 6C, FIG. 6D).

Further study of plant water content, membrane integrity and chlorophyll content revealed that WT and TG plants had similar cell membrane integrity level under normal condition. However, after 2 days of heat treatment, the cell membrane damage elicited by heat stress was significantly more severe in WT plant than that in the AsHSP17-expressing TG plants (FIG. 6E). Moreover, both WT and TG plants displayed similar RWC under normal condition, whereas under heat stress, water loss in the AsHSP17-containing TG plants was significantly more than that in the control plant without AsHSP17 (FIG. 6F). Each column represents means: SE. Error bars represent SE. “*”, “**” or “***” indicate significant differences between transgenic and WT plants at P<0.05, 0.01 or 0.001, respectively by Student's t test. The ΔΔCt method was used for real time PCR analysis. Actin gene AtActin1 was used as the endogenous control. Error bars represent SE.

The chlorophyll a (FIG. 6G, FIG. 6J), chlorophyll b (FIG. 6H, FIG. 6K) and total chlorophyll (FIG. 6I, FIG. 6L) content in WT plants were significant higher than those in TG plants under both 0d and 2d after heat and salt-stress conditions, suggesting a reduced chlorophyll production in transgenic plants overexpressing AsHSP17.

Further analysis of AsHSP17 transgenic and control plants revealed that although they were not significantly different in transpiration rate (FIG. 6N) and stomatal conductance (FIG. 6O) under normal growth conditions, AsHSP17 transgenics exhibited significantly lower photosynthesis rate than controls without AsHSP17 (FIG. 6M). Expression of photosynthesis-related genes was examined in both AsHSP17 transgenic and control plants. As shown in FIG. 6P, compared to wild type controls, the expression levels of the genes encoding the photosystem I (PSI) component PSAF, the Rubisco activase RCA, the Rubisco large subunit rbcL were all decreased in AsHSP17 transgenic plants, whereas that of the gene encoding chlorophyll a/b binding protein CAB1 was slightly increased in transgenics. No significant difference in expression in the gene encoding photosystem II (PSII), PSBO-1 was observed between wild type and transgenic plants (FIG. 3F).

### Overexpression of AsHSP17 Increases Plants Sensitivity to Salt Stress

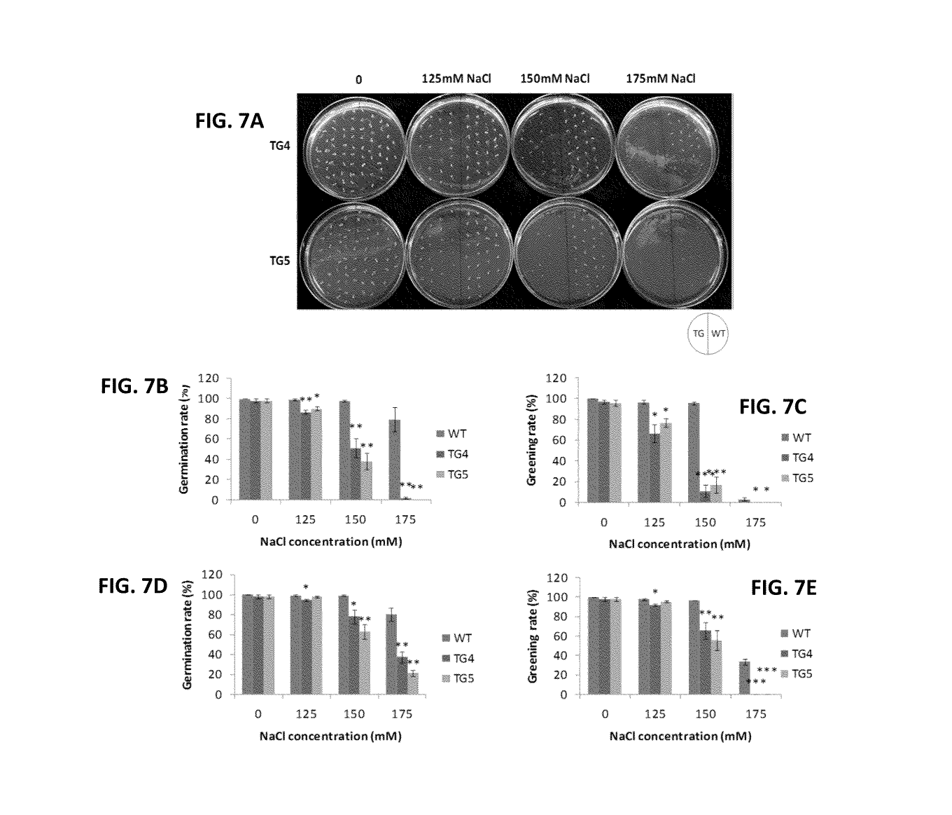

Plant response to salt stress was examined via germination rates in wild-type (WT) and two TG lines (TG4 and TG5) after 4d of treatment with 0, 125 mM, 150 mM and 175 mM NaCl. As shown in FIG. 7A, both WT and TG had similar germinating ability. However, under salt condition, especially with 150 mM and 175 mM NaCl treatments, much more WT plants germinated than TG plants. Each column in the figures represents means±SE. Error bars represent SE. “*”, “**” or “***” indicate significant differences between transgenic and WT plants at P<0.05, 0.01 or 0.001, respectively by Student's t test. Statistical analysis of germination and greening rates between WT and TG plants shows that both the germination and greening rates of WT plants were significantly higher than those of TG plants under the three different NaCl concentrations (FIG. 7B, FIG. 7C, FIG. 7D, FIG. 7E).

### Overexpression of AsHSP17 Leads to Hypersensitivity to Exogenous ABA During Germination and Post-Germinative Growth

The plant hormone ABA is a major mediator of plant development and various abiotic stresses (Leung and Giraudat, 1998; Finkelstein et al., 2002; Xiong et al., 2002; Zhu, 2002; Himmelbach et al., 2003; Yamaguchi-Shinozaki and Shinozaki, 2006).

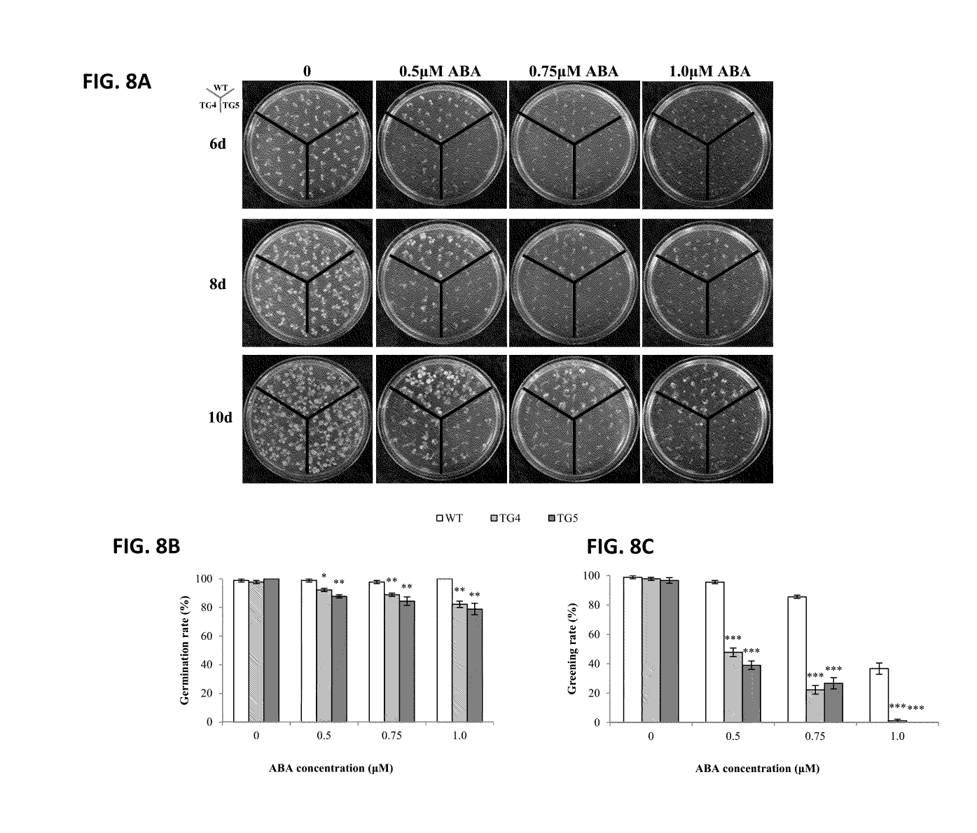

AsHSP17 expression is down-regulated in leaves, but significantly induced in roots by ABA. We therefore investigated whether AsHSP17 is involved in ABA-mediated inhibition of seed germination and/or post-germinative growth. We examined seed germination and post-germinative growth in both AsHSP17-expressing and wild type control plants treated with exogenous ABA (0, 0.5, 0.75 and 1 μM). As shown in FIG. 8A-8C, the germination rates of the AsHSP17 plants were significantly reduced on ABA-containing medium, but similar on ABA-free medium. The hypersensitivity of the AsHSP17-expressing plants to exogenous ABA compared to wild type control plants as measured by the cotyledone greening rates was also observed during the post-germinative growth phase, i.e., the cotyledon greening rates of the AsHSP17 plants were significantly lower than those of the controls under the three different concentrations of the ABA treatments (FIG. 8A, FIG. 8B, FIG. 8C). The data obtained indicate that AsHSP17 positively regulates ABA-induced inhibition of seed germination and early-stage seedling growth.

Transgenic Plants Overexpressing AsHSP17 are More Susceptible to Heat Stress than is Associated with Modified Expression of Heat-Responsive Genes

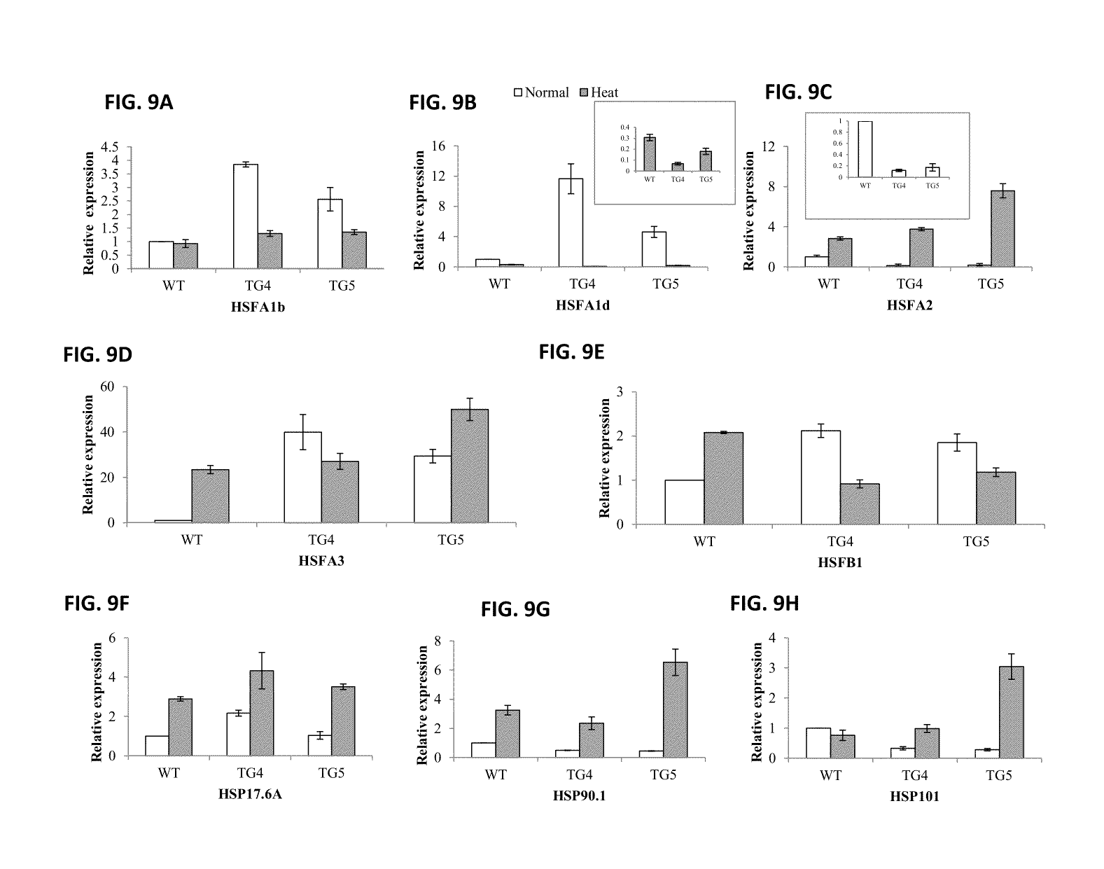

The attenuated plant response to heat stress mediated by AsHSP17 prompted examination of the expression level of some major HSF genes, HSFA1b, HSFA1d, HSFA2, HSFA3 and HSFB1 in wild type and AsHSP17 transgenic plants. The expression of both HSFA1b (FIG. 9A) and HSFA1d (FIG. 9B) in transgenic plants was significantly up-regulated compared to wild type controls under normal growth conditions. When exposed to heat stress, the expression of the HSFA1b gene in wild type controls remained unchanged, whereas its expression in transgenic plants dropped to the level comparable to that of the wild type controls (FIG. 9A). On the other hand, the expression of HSFA1d was down-regulated in both wild type control and transgenic plants, and the down regulation was more pronounced in transgenics than in the wild type controls (FIG. 9B). Class A2 HSF, HSFA2 is another dominant heat shock factor that determines heat stress response. Its expression level in transgenic plants was significantly lower than that in wild type controls under normal conditions. However, 4 h after exposure to heat stress, the expression of the HSFA2 was up-regulated in both wild type and transgenic plants, but the increase in HSFA2 expression was more pronounced in transgenic plants than in the wild type controls (FIG. 9C). HSFA3 was also examined, a HSF that regulates the expression of HSP-encoding genes (Schramm et al., 2008). Its expression was drastically enhanced (more than 20 folds) in AsHSP17 transgenic plants compared to wild type controls under normal growth conditions (FIG. 9D), but marginally changed in transgenics upon heat stress compared to the dramatic up-regulation (more than 20 folds) in the wild type controls (FIG. 9D). HSFB1 expression in the AsHSP17 transgenic plants was also enhanced compared with wild type controls. However, it was down-regulated in transgenic plants, but up-regulated in wild type controls in response to heat stress (FIG. 9E).

We then examined whether AsHSP17 impacts the expression of other heat shock protein genes, HSP17.6A, HSP90.1 and HSP101. As shown in FIG. 9F, compared with wild type control, a marginal increase in HSP17.6A expression was observed in the AsHSP17 transgenic plants, and its expression increased in both wild type and AsHSP17 transgenic plants after exposure to heat stress. The expression of HSP90.1 in the AsHSP17 transgenic plants was lower than that in wild type controls under normal conditions, and its expression was also significantly up-regulated in both wild type and transgenic plants after exposure to heat stress (FIG. 9G). Similarly, the expression of HSP101 in the AsHSP17 transgenic plants was significantly lower than that in wild type plants. Its expression was enhanced drastically in transgenic plants upon heat stress, whereas heat stress did not induce significant change in HSP101 expression in wild type controls (FIG. 9H).

Overexpression of AsHSP17 Increases Plant Sensitivity to Salt Stress that is Associated with Decreased Water Retention and Reduced Cell Membrane Integrity Compared to Salt-Stressed Wild Type Controls

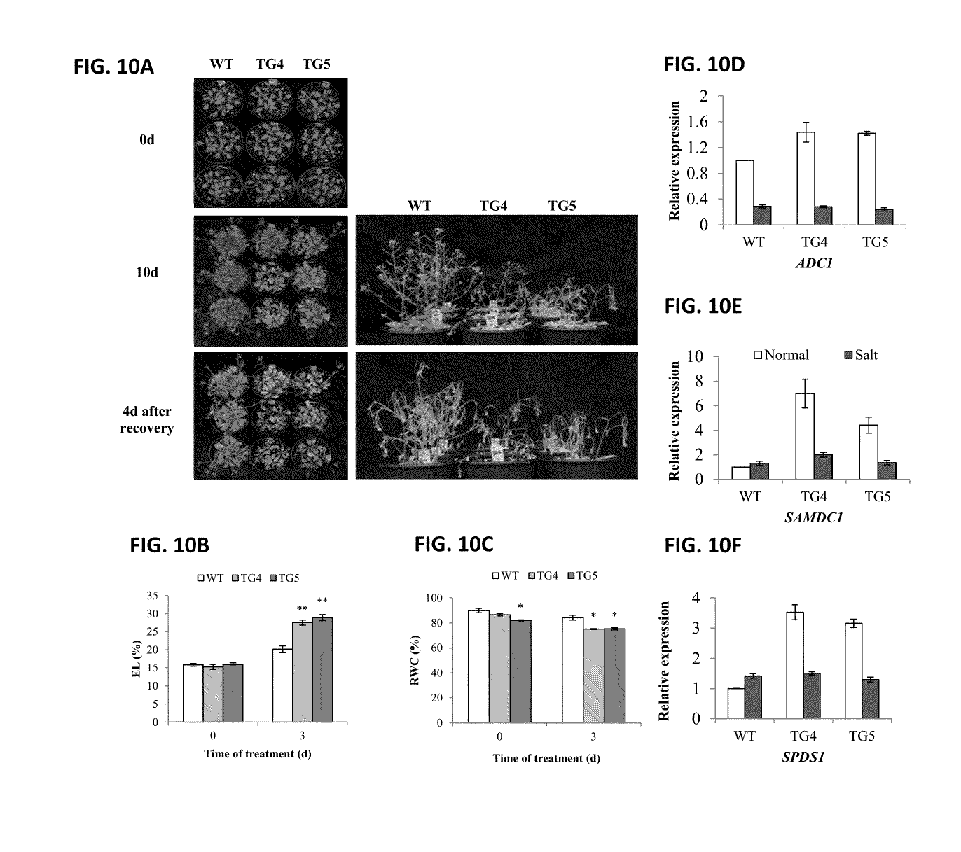

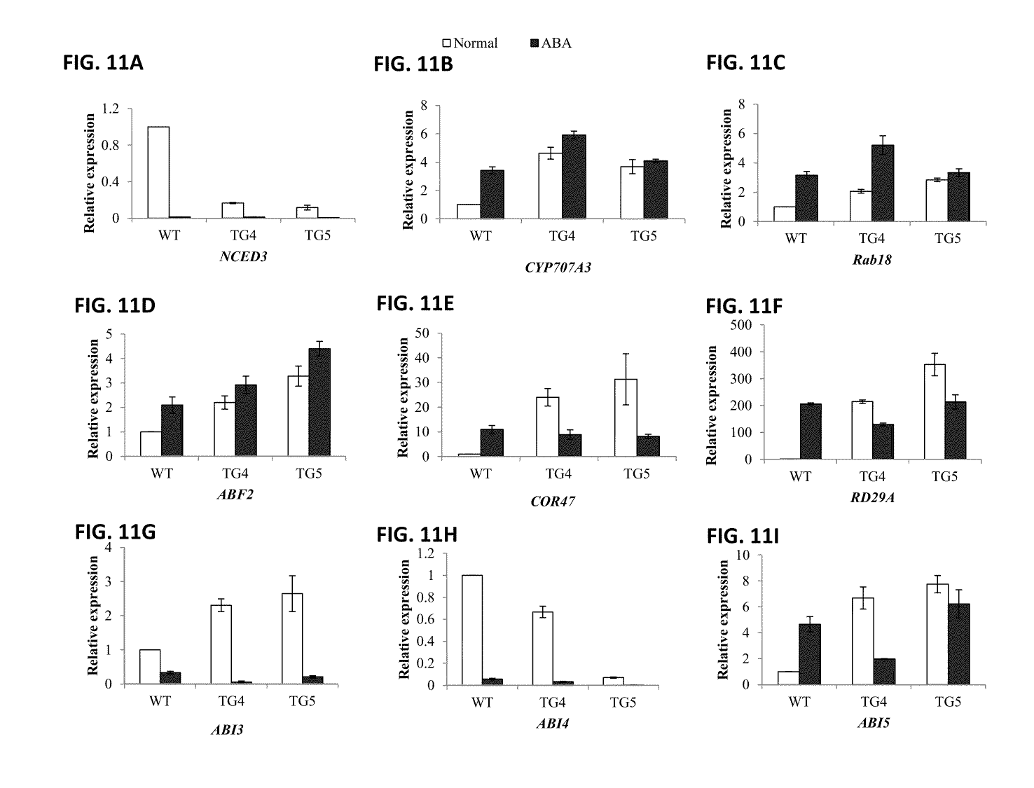

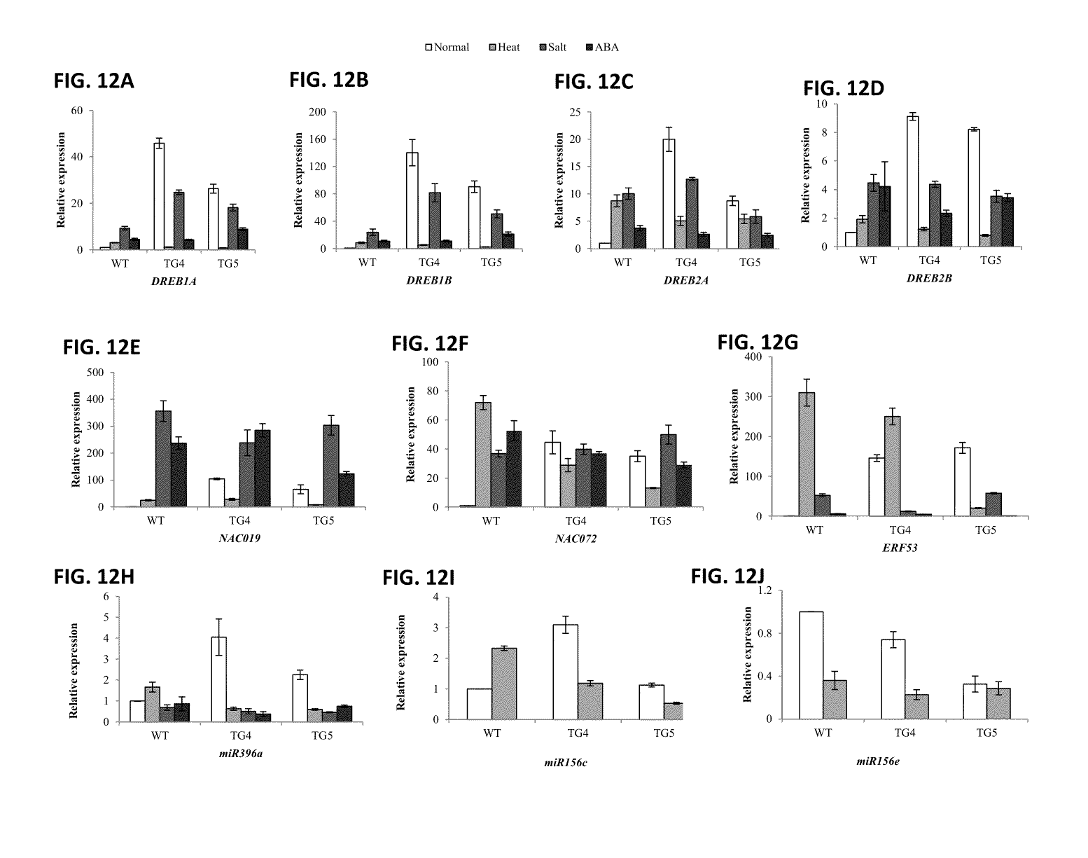

To study whether the overexpression of the AsHSP17 would impact plant response to salt stress, we examined performance of both wild type control and the AsHSP17-overexpressing transgenic plants grown under salinity stress (175 mM NaCl). As shown in FIG. 10A (middle panels), while no obvious stress symptom was observed in the non-transgenic wild type controls 10 d after salt treatment, salt-elicited damage appeared in most of the AsHSP17 transgenic plants, i.e., leaves turned yellowish and plants became wilted (FIG. 10A, middle panels), indicating decreased turgor pressure and accelerated onset of senescence compared with controls. The difference in stress symptom between transgenic and control plants became more pronounced during recovery. The AsHSP17 transgenic plants all withered 4 d after recovery from the stress, whereas wild type control plants just started showing stress symptom (FIG. 10A, bottom panels).

Further analysis of plant water status and cell membrane integrity revealed that although similar under normal growth conditions, significant differences between wild type control and the AsHSP17-expressing transgenic plants responding to salt stress were observed. The AsHSP17 transgenic plants exhibited significantly more rapid water loss (lower RWC) (FIG. 10B) and more severe cell membrane damage (higher EL) (FIG. 10C) than wild type controls 3 d after salt stress, suggesting a reduced water retention capacity and a decreased cell membrane integrity in transgenic plants resulting from AsHSP17 overexpression.

To explain the performance of the AsHSP17-overexpressing transgenic plants under salt stress conditions, we assessed salt response genes in both wild type control and the AsHSP17 transgenic plants. We first investigated whether AsHSP17 alters the biosynthesis of polyamines (PAs), the growth regulators ubiquitously present in all living cells and known to play an important role in plant adaptive response to environmental adversity including salt stress (Alcfzar et al., 2010; Wimalaskera et al., 2011). To do this, we examined some key genes of PA biosynthesis. As shown in FIG. 10D, FIG. 10E, and FIG. 10F, the expression of ADC1, a key gene for the biosynthesis of one of the most common free PAs, putrescine in the AsHSP17 transgenic plants was slightly higher than that in the wild type controls under normal conditions (FIG. 10D). After the plant exposure to salt stress for 4 h, ADC1 was significantly down regulated in both wild type and transgenic plants, and there was no significant difference in ADC1 expression level between them (FIG. 10D). Further assessment of two additional key genes, SAMCD1 and SPDS1 for the biosynthesis of the other two common free PAs, spermine and spermidine revealed that under normal conditions, the mRNA levels of the SAMCD1 and SPDS1 were significantly increased in the AsHSP17 transgenic plants compared with wild type controls (FIG. 10E, FIG. 10F). In contrast, upon salt stress, the expression of these two genes in transgenic plants dropped dramatically to the level comparable to that in wild type controls, which was slightly increased from that of the non-stressed conditions (FIG. 10E, FIG. 10F). Each column represents means±SE. Error bars represent SE. “*” or “**” indicate significant differences between transgenic and WT plants at P<0.05 or 0.01, respectively by Student's t test. The ΔΔCt method was used for real time PCR analysis. Actin gene AtActin1 was used as the endogenous control. Error bars represent SE.

sHSP17 Affects ABA Production and is Involved in ABA-Mediated Signaling

The results showing ABA-regulated AsHSP17 expression (FIG. 4E) and the observations that AsHSP17-expressing transgenic plants exhibited increased sensitivity to ABA and salt stresses in seed germination and post-germinative growth (FIGS. 7A-7E, FIGS. 8A-8C), but decreased resistance to heat (FIGS. 6A-6N) and salinity stresses (FIGS. 10A-10F) prompted us to investigate whether or not AsHSP17 affects ABA production and/or participates in ABA-mediated signaling. To do this, we first examined the expression of genes involved in ABA biosynthesis and catabolism. The 9-cis epoxycarotenoid dioxygenase (NCED) encoded by AtNCED3 gene catalyzes the limiting step of ABA biosynthesis (Roychoudhury, 2013). As shown in FIG. 11A, the expression of AtNCED3 in transgenic plants was significantly down-regulated compared to wild type controls under normal conditions. When treated with ABA, the expression of the AtNCED3 was significantly reduced in both wild type control and transgenic plants, and the reduction was more pronounced in transgenics than in the wild type controls (FIG. 11A). CYP707A3, encoded by the AtCYP707A3 gene, is the major ABA-catabolizing enzyme (Roychoudhury, 2013). As shown in FIG. 11B, CYP707A3 expression in transgenic plants was significantly higher than that in wild type controls under both normal and ABA treatment conditions. ABA treatment stimulated CYP707A3 expression, and the enhanced CYP707A3 expression by ABA was more significant in wild type control (3.4-fold) than in transgenic plants (<1.5-fold). The difference in AtNCED3 and CYP707A3 expression between AsHSP17 transgenic and wild type control plants suggests that overexpression of AsHSP17 may impair ABA biosynthesis and accumulation in plants.

Next, we analyzed expression of genes involved in ABA signaling pathway. As shown in FIG. 11C, FIG. 11D, Rab18 and ABF2 were both induced by ABA in wild type and transgenic plants and their expression in transgenic plants was significantly higher than that in wild type controls under both conditions with and without ABA treatment. Interestingly, COR47 (FIG. 11E) and RD29A (FIG. 11F), the other two genes involved in ABA signaling pathway exhibited significantly enhanced expression in the AsHSP17 transgenic plants compared to wild type controls under normal conditions (>20-fold and 200-fold, respectively). Upon 4 h of ABA treatment, however, their expression was strongly up-regulated in wild type controls to the level higher than or comparable to that in transgenic plants, which was down-regulated compared to what was observed under normal conditions.

We also examined expression of ABA-responsive genes, ABI3 (FIG. 11G), ABI4 (FIG. 11H), and ABI5 (FIG. 11I) in transgenic and wild type control plants under normal conditions and ABA treatment. As shown in FIG. 11G and FIG. 11I, the expression of ABI3 and ABI5 in transgenic plants was higher than that in wild type controls. ABA treatment resulted in down-regulated ABI3 expression in both wild type and transgenic plants, and this down-regulation was significantly more pronounced in transgenics than in wild type controls (FIG. 11G). However, ABI5 expression was up-regulated in wild type control, but down-regulated in transgenic plants by ABA (FIG. 11I). ABI4 expression was down-regulated in wild type control and transgenic plants under both normal conditions and ABA treatment (FIG. 11H). These results, together with the observations about the AsHSP17-mediated responsiveness to heat and salt stress suggest that AsHSP17 impacts ABA biosynthesis and is involved in ABA-mediated signaling to attenuate plant response to abiotic stress.

The Impact of AsHSP17 on the Expression of Other Genes Encoding Stress-Related Transcription Factors and miRNAs

To investigate whether AsHSP17 impacts important stress-related transcription factors, we studied the expression of the four dehydration responsive element-binding protein (DREB) transcription factor genes, DREBIA, DREBIB, DREB2A and DREB2B as well as NAC019, NAC072 and ERF53 known to be involved in plant response to abiotic stress (Lata and Prasad, 2011; Hickman et al., 2013; Guan et al., 2014; Hsieh et al., 2013). As demonstrated in FIG. 12A-FIG. 12D, under normal conditions, the expression levels of DREB1A (FIG. 12A), DREB1B (FIG. 12B), DREB2A (FIG. 12C) and DREB2B (FIG. 12D) in the AsHSP17 transgenic plants were all greatly enhanced compared to those in wild type controls (from 8-fold in DREB2B to approximately 150-fold in DREB1B expression). Strikingly, upon heat, salt or ABA treatment, the expression levels of these four DREB transcription factor genes, especially those of the DREB1A and DREB1B, were dramatically reduced in transgenic plants, whereas their expression levels in wild type plants were all increased. Similarly, under normal conditions, the expression of other transcription factor genes, NAC019 (FIG. 12E), NAC072 (FIG. 12F) and ERF53 (FIG. 12G) in the AsHSP17 transgenic plants was at least 40-fold more than that in the wild type controls. However, heat, salt or ABA treatment greatly induced the expression of the NAC019, NAC072 and ERF53 in wild type controls (an increase of approximately 350-fold in NAC019 expression under salt stress, and 300-fold in ERF53 expression under heat stress, for example) except that in the case of ERF53, no response to ABA treatment was observed in both wild type control and transgenic plants. On the contrary, NAC019 expression in transgenic plants was down-regulated by heat, but slightly up-regulated by salt and ABA. Minor changes in NAC072 expression upon heat, salt or ABA treatment was observed in transgenic plants. For ERF53, it was slightly up-regulated in TG4, but down-regulated in TG5 by heat stress, and it was down-regulated in both TG4 and TG5 by salt stress. Minor changes in the expression of the NAC019, NAC072 and ERF53 genes under heat, salt or ABA treatment were observed in the AsHSP17 transgenic plants (less than 3.5 folds).

MicroRNAs (miRNAs) are another group of important regulators involved in plant abiotic stress response (Jones-Rhoades and Bartel, 2004; Sunkar and Zhu, 2004; Zhao et al., 2007; Ding et al., 2009; Yang et al., 2010; Wang et al., 2011; Zhou and Luo, 2013; Zhou et al., 2013). To investigate whether AsHSP17 impacts miRNA expression before and after the plant exposure to heat stress, we examined the expression profiles of miR156 and miR396 in wild type and the AsHSP17 transgenic plants. As shown in FIG. 12H-FIG. 12J, under normal conditions, miR396a and miR156c are both up regulated (FIG. 12H, FIG. 12I), whereas miR156e was down regulated in transgenic plants compared to the wild type controls (FIG. 12J). When exposed to heat, miR396a and miR156c are both down regulated in transgenic plants, but up regulated in wild type controls. However, miR165e was down regulated in both the transgenic and wild type control plants. The miR396a was also down regulated by salt or ABA treatment in both wild type control and transgenic plants.

It will be appreciated that the foregoing examples, given for purposes of illustration, are not to be construed as limiting the scope of this disclosure. Although only a few exemplary embodiments of the disclosed subject matter have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure. Further, it is recognized that many embodiments may be conceived that do not achieve all of the advantages of some embodiments, yet the absence of a particular advantage shall not be construed to necessarily mean that such an embodiment is outside the scope of the present disclosure.

What is claimed is:

1. An isolated nucleic acid molecule comprising one of the following:
a) a synthetic polynucleotide encoding a small heat shock protein as set forth in SEQ ID NO: 1;
b) a synthetic polynucleotide encoding a functional equivalent of SEQ ID NO: 1;
c) a polynucleotide that comprises the nucleic acid sequence as set forth in SEQ ID NO: 2;
d) a polynucleotide that comprises a nucleic acid sequence that is complementary to SEQ ID NO: 2;
e) a polynucleotide that hybridizes to SEQ ID NO: 2 under one of the following conditions:
(i) 7% sodium dodecyl sulfate (SDS), 0.5 M NaPO₄, 1 mM ethylenediamine tetraacetic acid (EDTA) at 50° C. with a final wash in 2× standard saline citrate (SSC), 0.1% SDS at 50° C.;
(ii) 7% SDS, 0.5 M NaPO₄, 1 mM EDTA at 50° C. with a final wash in 1×SSC, 0.1% SDS at 50° C.;
(iii) 7% SDS, 0.5 M NaPO₄, 1 mM EDTA at 50° C. with a final wash in 0.5×SSC, 0.1% SDS at 50° C.;
(iv) 7% SDS, 0.5 M NaPO₄, 1 mM EDTA at 50° C. with a final wash in 0.1×SSC, 0.1% SDS at 50° C.; and
(v) 7% SDS, 0.5 M NaPO₄, 1 mM EDTA at 50° C. with a final wash in 0.1×SSC, 0.1% SDS at 65° C.

2. An expression cassette, transgenic plant or progeny thereof, transgenic seed or progeny thereof comprising a polynucleotide of claim 1.

3. A method for modulating the abiotic stress response of a transgenic plant, the method including modifying the expression level of the small heat shock protein AsHSP17 (SEQ ID NO: 1) or a functional equivalent thereof in the plant wherein the small heat shock protein AsHSP17 (SEQ ID NO: 1) is heterologous to the plant.

4. The method of claim 3, wherein the method comprises down regulating the expression of the small heat shock protein.

5. The method of claim 3, wherein the plant is a monocot species.

6. The method of claim 5, wherein the plant is a turfgrass species, a switchgrass species, maize, rice, sorghum, barley, wheat, millet, oats, or sugarcane.

7. The method of claim 5, wherein the plant is of the Agrostis genus.

8. The method of claim 7, wherein the plant is of the Agrostis stoloniferus species.
